SEQUENCE LISTING (14 sequences)

SEQ ID NO: 1 - protein, 805 aa, Mus musculus
MMCQKFYVVLLHWEFLYVIAALNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHGMCTVLFMD

SEQ ID NO: 2 - DNA, 2418 bp, Mus musculus
atgatgtgtcagaaattctatgtggttttgttacactgggaatttctttatgtgatagctgcacttaacctggcatatccaatctctccctggaaatttaagttgttttgtggaccaccgaacacaaccgatgactcctttctctcacctgctggagccccaaacaatgcctcggctttgaagggggcttctgaagcaattgttgaagctaaatttaattcaagtggtatctacgttcctgagttatccaaaacagtcttccactgttgctttgggaatgagcaaggtcaaaactgctctgcactcacagacaacactgaagggaagacactggcttcagtagtgaaggcttcagtttttcgccagctaggtgtaaactgggacatagagtgctggatgaaaggggacttgacattattcatctgtcatatggagccattacctaagaaccccttcaagaattatgactctaaggtccatcttttatatgatctgcctgaagtcatagatgattcgcctctgcccccactgaaagacagctttcagactgtccaatgcaactgcagtcttcggggatgtgaatgtcatgtgccggtacccagagccaaactcaactacgctcttctgatgtatttggaaatcacatctgccggtgtgagttttcagtcacctctgatgtcactgcagcccatgcttgttgtgaaacccgatccacccttaggtttgcatatggaagtcacagatgatggtaatttaaagatttcttgggacagccaaacaatggcaccatttccgcttcaatatcaggtgaaatatttagagaattctacaattgtaagagaggctgctgaaattgtctcagctacatctctgctggtagacagtgtgcttcctggatcttcatatgaggtccaggtgaggagcaagagactggatggttcaggagtctggagtgactggagttcacctcaagtctttaccacacaagatgttgtgtattttccacccaaaattctgactagtgttggatcgaatgcttcttttcattgcatctacaaaaacgaaaaccagattatctcctcaaaacagatagtttggtggaggaatctagctgagaaaatccctgagatacagtacagcattgtgagtgaccgagttagcaaagttaccttctccaacctgaaagccaccagacctcgagggaagtttacctatgacgcagtgtactgctgcaatgagcaggcgtgccatcaccgctatgctgaattatacgtgatcgatgtcaatatcaatatatcatgtgaaactgacgggtacttaactaaaatgacttgcagatggtcacccagcacaatccaatcactagtgggaagcactgtgcagctgaggtatcacaggcgcagcctgtattgtcctgatagtccatctattcatcctacgtctgagcccaaaaactgcgtcttacagagagacggcttttatgaatgtgttttccagccaatctttctattatctggctatacaatgtggatcaggatcaaccattctttaggttcacttgactcgccaccaacgtgtgtccttcctgactccgtagtaaaaccactacctccatctaacgtaaaagcagagattactgtaaacactggattattgaaagtatcttgggaaaagccagtctttccggagaataaccttcaattccagattcgatatggcttaagtggaaaagaaatacaatggaagacacatgaggtattcgatgcaaagtcaaagtctgccagcctgctggtgtcagacctctgtgcagtctatgtggtccaggttcgctgccggcggttggatggactaggatattggagtaattggagcagtccagcctatacgcttgtcatggatgtaaaagttcctatgagagggcctgaattttggagaaaaatggatggggacgttactaaaaaggagagaaatgtcaccttgctttggaagcccctgacgaaaaatgactcactgtgtagtgtgaggaggtacgtggtgaagcatcgtactgcccacaatgggacgtggtcagaagatgtgggaaatcggaccaatctcactttcctgtggacagaaccagcgcacactgttacagttctggctgtcaattccctcggcgcttcccttgtgaattttaaccttaccttctcatggcccatgagtaaagtgagtgctgtggagtcactcagtgcttatcccctgagcagcagctgtgtcatcctttcctggacactgtcacctgatgattatagtctgttatatctggttattgaatggaagatccttaatgaagatgatggaatgaagtggcttagaattccctcgaatgttaaaaagttttatatccacggtatgtgtactgtacttttcatggattag

SEQ ID NO: 3 - protein, 318 aa, Mus musculus
QVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGP

SEQ ID NO: 4 - DNA, 954 bp, Mus musculus
caagtctttaccacacaagatgttgtgtattttccacccaaaattctgactagtgttggatcgaatgcttcttttcattgcatctacaaaaacgaaaaccagattatctcctcaaaacagatagtttggtggaggaatctagctgagaaaatccctgagatacagtacagcattgtgagtgaccgagttagcaaagttaccttctccaacctgaaagccaccagacctcgagggaagtttacctatgacgcagtgtactgctgcaatgagcaggcgtgccatcaccgctatgctgaattatacgtgatcgatgtcaatatcaatatatcatgtgaaactgacgggtacttaactaaaatgacttgcagatggtcacccagcacaatccaatcactagtgggaagcactgtgcagctgaggtatcacaggcgcagcctgtattgtcctgatagtccatctattcatcctacgtctgagcccaaaaactgcgtcttacagagagacggcttttatgaatgtgttttccagccaatctttctattatctggctatacaatgtggatcaggatcaaccattctttaggttcacttgactcgccaccaacgtgtgtccttcctgactccgtagtaaaaccactacctccatctaacgtaaaagcagagattactgtaaacactggattattgaaagtatcttgggaaaagccagtctttccggagaataaccttcaattccagattcgatatggcttaagtggaaaagaaatacaatggaagacacatgaggtattcgatgcaaagtcaaagtctgccagcctgctggtgtcagacctctgtgcagtctatgtggtccaggttcgctgccggcggttggatggactaggatattggagtaattggagcagtccagcctatacgcttgtcatggatgtaaaagttcctatgagagggcct

SEQ ID NO: 5 - DNA, 9163 bp, Artificial sequence
  Synthetic sequence Vector pThy1-wp1
gtttccctaacagggccaagtgttttgagtaactgaaggtgggcatgatgcctgggaagcagaaacaagccaggcagatgcaccccttgccttgcttccgaagggctgcagtagcatggaaaacatggaaaacaaccaatccattccctttgctgatataacaggctccaaagccaaaacctgtcactggaggctcaagagcagatctccagccaagaggcaaaggaatgggggaagctggagggcctccctctggtttatccaggcttctgaaggttctagcaaagaaagggttacaaccttaaaaggagagcgtcccggggtatgggtagaagactgctccaccccgacccccagggtccctaaccgtcttttccttgggcgagtcagcccaatcacaggactgagagtgcctctttagtagcagcaagccacttcggacacccaaatggaacacctccagtcagccctcgccgaccaccccaccccctccatccttttccctccgcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgggatgcaagccttccccagctacagttttgggaaaggataccaaggcgctcctatatgggggcgcgggaacttggggaaagaaggtgctcccaggtcgaggtgggagaggaaggcagtgcggggtcacgggctttctccctgctaacggacgctttcgaagagtgggtgccggaggagaaccatgaggaaggacatcaaggacatcaaggacagcctttggtccccaagctcaaatcgctttagtggtgcgaatagagggaggaggtgggtggcaaactggagggagtccccagcgggtgacctcgtggctggctgggtgcggggcaccaggtaagaaaaccgcaatgttgcgggaggggactgggtggcaggcgcgggggaggggaaagctagaaaggatgcgagggagcggaggggggagggagcgggggaatctcaactggtagaggaaagttaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgctaggcttctccagccccagccccaattcttttctcagtgtccccttccctcccctgaatctcaagcccacactccctcctccataacccactgttatcaaatctaagtcatttgccacccaacaaccatcaggaggcggaagcagacgggaggagtttgagatcaacttgggctacatcacgagttccaagctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcggagactgtgcctctcttgggcaaggacaggctagacagcctgccggtgtgttgagctagggcactgtggggaaggcagagaacctgtgcagggcacgcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaactccttatttatccggtaaatgtcttttactctgctctcagggagctgaggcaggacattcctgagatacattgggagaggaatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctctgcctctctgcctctctgcctctctgcctctctctctgcctctctctgcctctctctgcccctctctgcccctctctgcccctctctgccgccctctgccttttgccctctgccctctgttctctggcctctgccctctccctgccttctccctctgcctctgcctcttgagtgctggaatcaaaggtgtgagctctataggtcttaagttccagaagaaacgtaatgaagtcacccagcaggaggtgctcagggacagcaagacacacacacccaggacactaggctcccacttccttggctttctctgagtggcaaaggaccttaggcagtgtcactccctaagagaaggggataaagagaggggctgaggtattcagtcatgtgctccgtggatctcaagccctcaaggtaaatggggacccacctgtcctaccagctggctgacctgtagctttccccaccacagaatccaagtcggaactcttggcactcgaggtccttcctctgcagaggtcttgcttctcccggtcagctgactccctccccaagtccttcaaatatctcagaacatggggagaaacggggaccttgtccctcctaaggaaccccagtgctgcatgccatcatcccccccaccctcgcccccacccccgccacttctccctccatgcataccactagctgtcattttgtactctgtatttattctagggctgcttctgattatttagtttgttctttccctggagacctgttagaacataagggcgtatggtgggtaggggaggcaggatatcagtcccctggggcgagttcctccctgccaaccaagccagatgcctgaaagagatatggatgagggaagttggactgtgcctgtacctggtacagtcatactctgttgaaagaatcatcggggaggggggggggctcaagaggggagagctctgctgagcctttgtggaccatccaatgaggatgagggcttagattctaccaggtcattctcagccaccacacacaagcgctctgccatcactgaagaagccccctagggccttgggccagggcacactcagtaaagatgcaggttcagtcagggaatgatggggaaaggggtaggaggtgggggagggatcaccccctcctctaaaacacgagcctgctgtctccaaaggcctctgcctgtagtgagggtggcagaagaagacaaggagccagaactctgactccaggatctaagtccgtgcaggaaggggatcctagaaccatctggttggacccagcttaccaagggagagcctttattcttcttttcccttgcccctctgtgccagcccctcttgctgtccctgatcccccagacagcgagagtcttgcaacctgcctcttccaagacctcctaatctcaggggcaggcggtggagtgagatccggcgtgcacactttttggaagatagctttcccaaggatcctctcccccactggcagctctgcctgtcccatcaccatgtataataccaccactgctacagcatctcaccgaggaaagaaaactgcacaataaaaccaagcctctggagtgtgtcctggtgtctgtctcttctgtgtcctggcgtctgtctcttctgtgttcttccaaggtcagaaacaaaaaccacacacttcaacctagatggctcggctgagcacttctgtgtgcagaaggtccaaccgactctgggtacccggccctccctattcccttgcctcctgtctcccgctttttatagctccctatgctgggcttctctggagagtgaaatctgtgcccgaaatcaatgcgcattctctctggtgagtcatctggcgacagcagtttgagttcacccgccaaacacatgggcccagctatgtagccgagactggctctggaagtgccagggactttggtgcataagtatgtaccatgcccttttttcacagtcctagctctgcagaagtgcagcctgaaggcctgtctgctgagaggacatgccctggagccctgaaacaggcacagtgggaggaggaacggaggatgacaggcatcaggccctcagtccaaaagcaaccacttgagaatgggctggagtacgaaacatggggtcccgtccctggatccctcctcaaagagtaataagtaaaatataaacaggtaccccaggccgttctgggtttgggttgtaatgggatccatttgcagagaactattgagacagcccagccgtactgtgacaggcaatgtgggggaggaggttgaatcacttggtatttagcatgaatagaataattccctgaacatttttcttaaacatccatatctaaattaccaccactcgctcccagtcttcctgcctttgcgccagcctcctgtctggccatgcctgaagaaggctggagaagccacccacctcaggccatgacactgccagccacttggcaggtgcagccaaacctgagctgtcccagaaagggacattctcaagacccaggcaccctgatcagcactgacttggagctacaagtgtcatgccagaaaagtctctaagaaaaccttttcagggaaaagggggtgactcaacaccgggcaagtttgggaagccccacccttcgagtgatggaagagcagataggaagcctcagaagagagacaccggcacccaggtaacgttcctcatgtggtctctgtcacactaggtgctcttccctggacatctccgtgaccacactctcagttcttagggagatgcgggtgctctctgaggctatctcaaaagtttgcagattctgaggcctagagtgactacagtcagcctaggaagccacagaggactgtggaccaggagggcagaagaggagaagggaagaaaaaccatcagataggacttgcaatgaaactaacccaagacaatcataatgcagacaggaatgttaaaggcgttcagcagctggccatgacacccatctgtccctctggccaagtcagcaagcctggaagacctgggactcctgcccatatgtcctaagctccccacccacccactcgttcactgtccttattctctctctaccttcagccacttagtttcctaccttaagtcctagaattgatcctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggatcgcggccgcgatccccgggcgagctcgaattcgtacgcgtttaaaccggtaattcagagaccgggaaccaaactagcctttaaaaaacataagtacaggagccagcaagatggctcagtgggtaaaggtgcctaccagcaagcctgacagcctgagttcagtccccacgaactacgtggtaggagaggaccaaccaactctggaaatctgttctgcaaacacatgctcacacacacacacacaaatagtataaacaattttaaatttcatttaaaaataatttgtaaacaaaatcattagcacaggttttagaaagagcctcttggtgacatcaagttgatgctgtagatggggtatcattcctgaggacccaaaaccgggtctcagcctttccccattctgagagttctctcttttctcagccactagctgaagagtagagtggctcagcactgggctcttgagttcccaagtcctacaactggtcagcctgactactaaccagccatgaagaaacaaggagtggatgggctgagtctgctgggatgggagtggagttagtaagtggccatggatgtaatgaccccagcaatgctggctaaaaggcatgcctcctttccttgtctggagacggaacgggagggatcatcttgtactcacagaagggagaacattctagctggttgggccaaaatgtgcaagttcacctggaggtggtggtgcatgcttttaactccagtactcaggaggcagggccaggtggatctctgtgagttcaagaccagcctgcactatggagagagttttgggacagccagagttacacagaaaaatcctggtggaaaatctgaaagaaagagagaaagaaagaaagaaagaaaggaagaaagaaagaaagagtggcaggcaggcaggcaggaggaaggaaggaaggaaggaaggaaggaaggaaggaaggaaggaaaataggtgcgacttcaagatccggagttacaagcagaatgcact

SEQ ID NO: 6 - protein, 167 aa, Mus musculus
MCWRPLCRFLWLWSYLSYVQAVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 7 - DNA, 504 bp, Mus musculus
atgtgctggagacccctgtgtcggttcctgtggctttggtcctatctgtcttatgttcaagcagtgcctatccagaaagtccaggatgacaccaaaaccctcatcaagaccattgtcaccaggatcaatgacatttcacacacgcagtcggtatccgccaagcagagggtcactggcttggacttcattcctgggcttcaccccattctgagtttgtccaagatggaccagactctggcagtctatcaacaggtcctcaccagcctgccttcccaaaatgtgctgcagatagccaatgacctggagaatctccgagacctcctccatctgctggccttctccaagagctgctccctgcctcagaccagtggcctgcagaagccagagagcctggatggcgtcctggaagcctcactctactccacagaggtggtggctttgagcaggctgcagggctctctgcaggacattcttcaacagttggatgttagccctgaatgctga

SEQ ID NO: 8 - protein, 1162 aa, Mus musculus
MMCQKFYVVLLHWEFLYVIAALNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGLYVIVPIIISSCVLLLGTLLISHQRMKKLFWDDVPNPKNCSWAQGLNFQKPETFEHLFTKHAESVIFGPLLLEPEPISEEISVDTAWKNKDEMVPAAMVSLLLTTPDPESSSICISDQCNSANFSGSQSTQVTCEDECQRQPSVKYATLVSNDKLVETDEEQGFIHSPVSNCISSNHSPLRQSFSSSSWETEAQTFFLLSDQQPTMISPQLSFSGLDELLELEGSFPEENHREKSVCYLGVTSVNRRESGVLLTGEAGILCTFPAQCLFSDIRILQERCSHFVENNLSLGTSGENFVPYMPQFQTCSTHSHKIMENKMCDLTV

SEQ ID NO: 9 - DNA, 3489 bp, Mus musculus
atgatgtgtcagaaattctatgtggttttgttacactgggaatttctttatgtgatagctgcacttaacctggcatatccaatctctccctggaaatttaagttgttttgtggaccaccgaacacaaccgatgactcctttctctcacctgctggagccccaaacaatgcctcggctttgaagggggcttctgaagcaattgttgaagctaaatttaattcaagtggtatctacgttcctgagttatccaaaacagtcttccactgttgctttgggaatgagcaaggtcaaaactgctctgcactcacagacaacactgaagggaagacactggcttcagtagtgaaggcttcagtttttcgccagctaggtgtaaactgggacatagagtgctggatgaaaggggacttgacattattcatctgtcatatggagccattacctaagaaccccttcaagaattatgactctaaggtccatcttttatatgatctgcctgaagtcatagatgattcgcctctgcccccactgaaagacagctttcagactgtccaatgcaactgcagtcttcggggatgtgaatgtcatgtgccggtacccagagccaaactcaactacgctcttctgatgtatttggaaatcacatctgccggtgtgagttttcagtcacctctgatgtcactgcagcccatgcttgttgtgaaacccgatccacccttaggtttgcatatggaagtcacagatgatggtaatttaaagatttcttgggacagccaaacaatggcaccatttccgcttcaatatcaggtgaaatatttagagaattctacaattgtaagagaggctgctgaaattgtctcagctacatctctgctggtagacagtgtgcttcctggatcttcatatgaggtccaggtgaggagcaagagactggatggttcaggagtctggagtgactggagttcacctcaagtctttaccacacaagatgttgtgtattttccacccaaaattctgactagtgttggatcgaatgcttcttttcattgcatctacaaaaacgaaaaccagattatctcctcaaaacagatagtttggtggaggaatctagctgagaaaatccctgagatacagtacagcattgtgagtgaccgagttagcaaagttaccttctccaacctgaaagccaccagacctcgagggaagtttacctatgacgcagtgtactgctgcaatgagcaggcgtgccatcaccgctatgctgaattatacgtgatcgatgtcaatatcaatatatcatgtgaaactgacgggtacttaactaaaatgacttgcagatggtcacccagcacaatccaatcactagtgggaagcactgtgcagctgaggtatcacaggcgcagcctgtattgtcctgatagtccatctattcatcctacgtctgagcccaaaaactgcgtcttacagagagacggcttttatgaatgtgttttccagccaatctttctattatctggctatacaatgtggatcaggatcaaccattctttaggttcacttgactcgccaccaacgtgtgtccttcctgactccgtagtaaaaccactacctccatctaacgtaaaagcagagattactgtaaacactggattattgaaagtatcttgggaaaagccagtctttccggagaataaccttcaattccagattcgatatggcttaagtggaaaagaaatacaatggaagacacatgaggtattcgatgcaaagtcaaagtctgccagcctgctggtgtcagacctctgtgcagtctatgtggtccaggttcgctgccggcggttggatggactaggatattggagtaattggagcagtccagcctatacgcttgtcatggatgtaaaagttcctatgagagggcctgaattttggagaaaaatggatggggacgttactaaaaaggagagaaatgtcaccttgctttggaagcccctgacgaaaaatgactcactgtgtagtgtgaggaggtacgtggtgaagcatcgtactgcccacaatgggacgtggtcagaagatgtgggaaatcggaccaatctcactttcctgtggacagaaccagcgcacactgttacagttctggctgtcaattccctcggcgcttcccttgtgaattttaaccttaccttctcatggcccatgagtaaagtgagtgctgtggagtcactcagtgcttatcccctgagcagcagctgtgtcatcctttcctggacactgtcacctgatgattatagtctgttatatctggttattgaatggaagatccttaatgaagatgatggaatgaagtggcttagaattccctcgaatgttaaaaagttttatatccacgataattttattcccatcgagaaatatcagtttagtctttacccagtatttatggaaggagttggaaaaccaaagataattaatggtttcaccaaagatgctatcgacaagcagcagaatgacgcagggctgtatgtcattgtacccataattatttcctcttgtgtcctactgctcggaacactgttaatttcacaccagagaatgaaaaagttgttttgggacgatgttccaaaccccaagaattgttcctgggcacaaggactgaatttccaaaagcctgaaacatttgagcatctttttaccaagcatgcagaatcagtgatatttggtcctcttcttctggagcctgaacccatttcagaagaaatcagtgtcgatacagcttggaaaaataaagatgagatggtcccagcagctatggtctcccttcttttgaccacaccagaccctgaaagcagttctatttgtattagtgaccagtgtaacagtgctaacttctctgggtctcagagcacccaggtaacctgtgaggatgagtgtcagagacaaccctcagttaaatatgcaactctggtcagcaacgataaactagtggaaactgatgaagagcaagggtttatccatagtcctgtcagcaactgcatctccagtaatcattccccactgaggcagtctttctctagcagctcctgggagacagaggcccagacatttttccttttatcagaccagcaacccaccatgatttcaccacaactttcattctcggggttggatgagcttttggaactggagggaagttttcctgaagaaaatcacagggagaagtctgtctgttatctaggagtcacctccgtcaacagaagagagagtggtgtgcttttgactggtgaggcaggaatcctgtgcacattcccagcccagtgtctgttcagtgacatcaggatcctccaggagagatgctcacactttgtagaaaataatttgagtttagggacctctggtgagaactttgtaccttacatgccccaatttcaaacctgttccacgcacagtcacaagataatggagaataagatgtgtgacttaactgtgtaa

SEQ ID NO: 10 - DNA, 2152 bp, Artificial sequence
  Synthetic sequence  Nucleotide sequence that includes the Thy-1 promoter sequence.
ggtgacctcgtggctggctgggtgcggggcaccaggtaagaaaaccgcaatgttgcgggaggggactgggtggcaggcgcgggggaggggaaagctagaaaggatgcgagggagcggaggggggagggagcgggggaatctcaactggtagaggaaagttaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgctaggcttctccagccccagccccaattcttttctcagtgtccccttccctcccctgaatctcaagcccacactccctcctccataacccactgttatcaaatctaagtcatttgccacccaacaaccatcaggaggcggaagcagacgggaggagtttgagatcaacttgggctacatcacgagttccaagctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcggagactgtgcctctcttgggcaaggacaggctagacagcctgccggtgtgttgagctagggcactgtggggaaggcagagaacctgtgcagggcacgcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaactccttatttatccggtaaatgtcttttactctgctctcagggagctgaggcaggacattcctgagatacattgggagaggaatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctctgcctctctgcctctctgcctctctgcctctctctctgcctctctctgcctctctctgcccctctctgcccctctctgcccctctctgccgccctctgccttttgccctctgccctctgttctctggcctctgccctctccctgccttctccctctgcctctgcctcttgagtgctggaatcaaaggtgtgagctctataggtcttaagttccagaagaaacgtaatgaagtcacccagcaggaggtgctcagggacagcaagacacacacacccaggacactaggctcccacttccttggctttctctgagtggcaaaggaccttaggcagtgtcactccctaagagaaggggataaagagaggggctgaggtattcagtcatgtgctccgtggatctcaagccctcaaggtaaatggggacccacctgtcctaccagctggctgacctgtagctttccccaccacagaatccaagtcggaactcttggcactcgag

SEQ ID NO: 11 - DNA, 34 bp, Artificial sequence
  Synthetic sequence Oligonucleotide wh65
gcgaagcttatgatgtgtcagaaattctatgtgg

SEQ ID NO: 12 - DNA, 34 bp, Artificial sequence
  Synthetic sequence Oligonucleotide wh66
gcggtcgacctaatccatgaaaagtacagtacac

SEQ ID NO: 13 - DNA, 20 bp, Artificial sequence
  Synthetic sequence Oligonucleotide wh151
gactacaaggacgatgacga

SEQ ID NO: 14 - DNA, 20 bp, Artificial sequence
  Synthetic sequence Oligonucleotide wh152
tcccctttcatccagcactc

(19) United States
(12) Patent Application Publication
(10) Pub. No.: US 2009/0031434 A1
(43) Pub. Date: Jan. 29, 2009

(54) ANIMAL MODELS FOR OBESITY AND NEURODEGENERATIVE DISEASES

(76) Inventor: Weiping Han, (US)

Correspondence Address: SWANSON & BRATSCHUN, L.L.C., 8210 SOUTHPARK TERRACE, LITTLETON, CO, 80120, US

(21) Appl. No.: 11/828,832

(22) Filed: Jul. 26, 2007

(51) Int. Cl. A01K 67/00; C07H 21/04

(52) U.S. Cl. 800/3; 536/23.5; 800/12; 800/25; 800/9

(57) ABSTRACT

A transgenic non-human animal is disclosed, the animal having a nucleic acid inserted in its genome, wherein the presence of the inserted nucleic acid in the genome of the animal results in expression of an agent, which agent is encoded by a nucleotide sequence in the genome of the animal, and wherein the agent inhibits the ability of a leptin to activate an Ob-Rb receptor. Uses of the animal and methods of identifying compounds using the animal are also disclosed.

### FIELD OF THE INVENTION

The present invention relates to transgenic non-human animal models for obesity and neurodegenerative disease, such as Alzheimer's disease. It also relates to use of the animal models to identify and obtain new therapeutic compounds for treating obesity and neurodegenerative diseases.

### BACKGROUND TO THE INVENTION

Leptin is the hormone product of the ob (obese) gene and it plays a role in regulating energy intake and expenditure, including regulation of appetite and metabolism. It is produced by adipose tissue and its actions are mediated via the Ob receptor, which is located in the hypothalamus of the brain and peripheral tissues, including liver and adipose tissue (Ahima et al., Robinson et al., Baile et al.).

There are five isoforms of the Ob receptor. The long form, Ob-Rb, is the only one that is capable of transmitting leptin signaling. Three short forms, OB-Ra, OB-Rc, and OB-Rd, are poorly studied and their functions are not clear. OB-Ra has been proposed to serve as a transporter to facilitate leptin access to the brain through the blood brain barrier. The fifth isoform, OB-Re, does not contain a transmembrane region, and is present in the plasma in a soluble form, thus the name soluble leptin receptor (SLR) (Ahima et al.).

Leptin resistance in animals has been shown to lead to severe obesity, and diet-induced obese animals have also been shown to be resistant to leptin. The cause of obesity is complex, involving both genetic and environmental factors.

Alzheimer's disease is a neurodegenerative disease that affects millions of people world wide. Alzheimer disease is associated with the accumulation of β-amyloid products (Price et al., Stokin et al.). Disruption of leptin signaling has been shown to be associated with increased β-secretase activity, which leads to the accumulation of β-amyloid products (Fewlass et al.).

There exist a number of mouse models mimicking neurodegenerative diseases, including Alzheimer's disease (AD) and Parkinson disease (PD) (Heitz et al., Bonini et al.). These disease models recreate the hallmarks and/or symptoms of the respective diseases, e.g. plaques and neurofibrillary tangles for AD, tremors and motor defects for PD. However, a suitable animal model based on disruption of key signaling pathways in neurodegeneration is not yet available.

Obesity and Alzheimer's diseases are conditions that debilitate millions of people world wide. As yet there is no cure for Alzheimer's disease. New treatments for both of these conditions are urgently needed.

### SUMMARY OF THE INVENTION

The inventor has had the surprising insight that Ob-Re presents a link between obesity and neurodegeneration. Without being bound by theory, the inventor believes that Ob-Re binds to leptin, and may thereby be used to chelate leptin and prevent it from interacting with the Ob-Rb receptor such that downstream signaling mediated by the Ob-Rb receptor does not take place. The inventor considers the disruption of the leptin signaling pathway will prevent the brain from registering satiation, thereby leading to obesity. Furthermore, the inventor considers that preventing leptin from interacting with the Ob-Re receptor will lead to an increase in β-secretase activity, thereby leading to accumulation of β-amyloid products and neurodegeneration.

Without being bound by theory, the inventor also believes that the effect of an increase in β-secretase activity will enhance γ-secretase activity. This may further result in increased proteolytic processing of Ob-R to generate more Ob-Re, thereby forming a positive feedback mechanism to further down-regulate the leptin signaling pathway.

As far as the inventor is aware, this is a new insight. The inventor is unaware of any previous suggestion that Ob-Re is a causal factor in both obesity and neurodegeneration. The inventor is also unaware of any previous suggestion linking a deficiency in leptin signaling with Ob-Re, and nor are they aware of any previous suggestion of a role for β- and γ-secretases in generating Ob-Re.

Following on from this insight, the inventor has created a transgenic mouse model that over-expresses Ob-Re in the nervous system. This model re-creates the underlying causes of obesity and Alzheimer's disease. As the transgenic mouse over-expresses soluble Ob-Re in the nervous system, the excess Ob-Re binds leptin and prevents it binding to the cell-membrane bound Ob-Rb and therefore prevents activation of the leptin:Ob-Rb signaling pathway.

Importantly, this animal model is not a genomic “knock-out” animal model, i.e. all natural genes and their promoters remain intact. In particular, leptin will still be expressed, and the animal is still capable of expressing all of the signaling molecules involved in the leptin signaling pathway.

This animal model provides a valuable tool for identifying and testing new treatments for obesity and neurodegenerative disorders such as Alzheimer's disease. In addition, the animal is useful in identifying disease-causing molecules that may be targets for drug developments.

Thus, the invention broadly relates to a transgenic non-human animal model in which binding of leptin to the Ob-Rb receptor is repressed, prevented or inhibited (whether wholly or partly), and to use of such animal models for identifying compounds for treating obesity and neurodegenerative conditions.

In a first aspect of the invention, there is provided a transgenic non-human animal having a nucleic acid inserted in its genome, wherein the presence of the inserted nucleic acid in the genome of the animal results in expression of an agent, which agent is encoded by a nucleotide sequence in the genome of the animal, and wherein the agent inhibits the ability of a leptin to activate an Ob-Rb receptor.

The agent may inhibit, partially or wholly, the ability of a leptin to activate the Ob-Rb receptor by binding to the leptin. For example, the presence of the agent may reduce and/or repress the ability of leptin to activate the Ob-Rb receptor compared to the absence of the agent. In particular, the agent may prevent the leptin from activating the Ob-Rb receptor.

In a further aspect, there is provided a transgenic non-human animal having a nucleic acid inserted in its genome, wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor.

The agent may inhibit, partially or wholly, binding of the leptin to the Ob-Rb receptor. For example, the presence of the agent may reduce and/or repress the ability of leptin to bind the Ob-Rb receptor compared to the absence of the agent. Preferably, the agent chelates leptin, e.g. it binds tightly to leptin. In particular, binding of the agent to the leptin may prevent the leptin from binding, e.g. docking, with the Ob-Rb receptor. For example, the agent may inhibit the binding of leptin to the Ob-Rb receptor by steric hindrance. In particular, the agent may bind to leptin such that the leptin is unable to bind to the Ob-Rb receptor in a configuration that activates the Ob-Rb receptor. The agent may bind leptin at or near the site on leptin that binds the Ob-Rb receptor.

Binding of the agent to the leptin may inhibit, e.g. reduce and/or repress, the ability of the leptin to activate the Ob-Rb receptor. Inhibiting leptin from binding and/or activating the Ob-Rb receptor will preferably inhibit the signaling cascade triggered by the Ob-Rb receptor. For example, binding of the agent to leptin will have the effect of inhibiting, e.g. reducing and/or repressing, leptin mediated signaling. The agent may induce full or partial leptin resistance. Leptin resistance is, for example, an inability of an animal to respond to leptin.

Preferably, the inserted nucleic acid is present at a position in the genome of the animal, at which position the nucleic acid does not occur naturally. This means, for example, that the genome of the animal is engineered to include additional nucleic acid at a particular position, which is not present at that position in the wild type animal. Although the animal may have similar or identical endogenous nucleic acid (i.e. a nucleic acid that occurs naturally in the genome of the wild-type animal) elsewhere in its genome, the genome does not naturally include the additional nucleic acid at that position of insertion.

Where the genome of the animal includes an endogenous nucleotide sequence encoding a suitable agent, e.g. Ob-Re, the inserted nucleic acid may comprise a regulatory element, e.g. a promoter. The regulatory element may be inserted in the genome of the animal such that it is operably linked to the endogenous nucleotide sequence encoding the agent. The animal may then express the agent under control of the inserted regulatory element.

Where the genome of the animal includes a suitable endogenous regulatory element, e.g. a promoter, for a nucleotide sequence encoding the agent, the inserted nucleic acid may comprise a nucleotide sequence encoding the agent, without comprising a regulatory element. The nucleic acid may be inserted in the genome of the animal such that the nucleotide sequence is operably linked to the regulatory element. The animal may then express the agent.

In addition, or alternatively, the inserted nucleic acid may comprise a nucleotide sequence encoding an agent, and/or it may comprise a regulatory element, irrespective of whether the genome of the animal comprises an endogenous nucleotide sequence encoding a suitable agent, and/or a suitable endogenous regulatory element for an agent.

The nucleic acid sequence encoding the agent may or may not be an endogenous nucleic acid sequence. Likewise, the regulatory element may or not be an endogenous regulatory element. The regulatory element may be operably linked to the nucleotide sequence encoding the agent, e.g. such that the animal expresses the agent when the nucleic acid is inserted into the genome of the animal. Where the promoter and the nucleic acid sequence encoding the agent are both endogenous, the promoter is preferably not naturally operably linked to the nucleotide sequence, e.g. in the wild-type animal.

Preferably, the animal over-expresses the agent, e.g. it expresses the agent at a higher level compared to expression of any agent in the absence of the inserted nucleic acid. For example, the expression of the agent may be at least 1.5, 2, 2.5, 3, 3.5, 4, 4.5, or 5 times higher compared to expression in the absence of the inserted nucleic acid.

The inserted nucleic acid may encode a heterologous construct, e.g. an engineered DNA construct. The heterologous construct may include a nucleotide sequence encoding an agent and a regulatory element, e.g. a promoter, operably linked to the nucleotide sequence encoding the agent.

A transgenic animal expressing such an agent will preferably have a predisposition to obesity and/or Alzheimer's disease.

The ability of a leptin to activate an Ob-Rb receptor may be determined, for example, by an assay to detect phosphorylation of downstream targets, such as Jak2, STAT3, ERK, PI3K and AKt, e.g. by western blotting. Such assays are well-known to the person skilled in the art. Another way of determining the ability of a leptin to activate an Ob-Rb receptor is to use a luciferase assay system, which is described in more detail below.

The ability of a leptin to bind Ob-Rb may be determined, for example, by measuring binding of leptin to cells that express the Ob-Rb receptor, e.g. using ELISA. Such methods are well known to the person skilled in the art. The agent may reduce the ability of a leptin to bind the Ob-Rb receptor by at least 20, 30, 40, 50, 60, 70, 75, 80, 85, 90, 95, 96, 97, 98, 99, or 100 percent compared to the ability of a leptin to bind the Ob-Rb receptor in the absence of the agent.

Preferably, the agent specifically binds to leptin, e.g. it selectively binds to leptin. The agent may bind leptin with high affinity, e.g. with a Kd of less than 1 μM. Preferably, binding of the agent to the leptin prevents, e.g. substantially prevents, binding of the leptin to the Ob-Rb receptor.

The agent preferably comprises the binding domain of a leptin receptor. For example, the Ob gene isoforms, e.g. Ob-Ra, Ob-Rb, Ob-Rc, Ob-Rd, and Ob-Re comprise leptin binding domains. In particular, the Ob-Re isoform of the Ob receptor binds tightly to leptin. It has been reported that residues 323-640 of the leptin receptors are responsible for leptin binding. This domain contains the second segment of cytokine receptor domain/fibronectin type 3 domain (residues 428-635). SEQ ID NO: 3 shows amino acids 323-640 of Ob-Re and SEQ ID NO: 4 shows the nucleotide sequence encoding amino acids 323-640 of Ob-Re.

The amino acid sequence of the Ob-Re receptor is shown in SEQ ID NO: 1 and the nucleotide sequence encoding the Ob-Re receptor is shown in SEQ ID NO: 2.

The agent may, for example, comprise the amino acid sequence of the Ob-Re receptor or the leptin binding domain of the Ob-Re receptor, an amino acid sequence sharing a degree of sequence homology with the Ob-Re receptor or the leptin binding domain of the Ob-Re receptor, a fragment of the Ob-Re receptor, or an amino acid sequence sharing a degree of sequence homology with a fragment of the Ob-Re receptor. Preferably, the agent is the Ob-Re receptor.

In particular, the agent may comprise:
(i) the amino acid sequence of SEQ ID NO: 1 or SEQ ID NO: 3,
(ii) an amino acid sequence sharing at least 70 percent sequence identity with the amino sequence of SEQ ID NO: 1 or SEQ ID NO: 3,
(iii) a fragment of the amino acid sequence of SEQ ID NO: 1 or SEQ ID NO: 3, which fragment has at least 10 contiguous amino acids of the amino acid sequence of SEQ ID NO: 1 or SEQ ID NO: 3, or
(iv) an amino acid sequence sharing at least 70 percent sequence identity with the fragment defined in (iii), wherein the amino acid sequence has at least 10 amino acids, and less than 805 amino acids.

Alternatively, the agent may consist of an amino acid sequence defined in (i), (ii), (iii), (iv), or it may consist essentially of an amino acid sequence defined in (i), (ii), (iii), or (iv).

Alternatively, the agent may be an antibody that is specific for leptin, i.e. the antibody may selectively bind to leptin with high affinity.

Preferably, the agent inhibits the ability of a leptin to activate the Ob-Rb receptor and/or binding of a leptin to the Ob-Rb receptor.

Preferably the agent is not membrane bound, e.g. the agent does not include a transmembrane domain. The agent may be soluble, e.g. soluble in blood. For example, the agent may be in circulation, e.g. it may be circulated around the body by the vascular system of the animal. The agent may be expressed and subsequently secreted from a cell into the blood of the non-human animal. For example, the agent is an extracellular agent, e.g. the agent occupies intercellular spaces in the animal.

The inserted nucleic acid expressed by a cell of the transgenic non-human animal may comprise a nucleotide sequence that encodes any agent as defined in this specification.

For example, the inserted nucleic acid may comprise a nucleotide sequence that encodes a polypeptide comprising:
(i) the amino acid sequence of SEQ ID NO: 1 or SEQ ID NO: 3,
(ii) an amino acid sequence sharing at least 70 percent sequence identity with the amino sequence of SEQ ID NO: 1 or SEQ ID NO: 3,
(iii) a fragment of the amino acid sequence of SEQ ID NO: 1 or SEQ ID NO: 3, which fragment has at least 10 contiguous amino acids of the amino acid sequence of SEQ ID NO: 1 or SEQ ID NO: 3, or
(iv) an amino acid sequence sharing at least 70 percent sequence identity with the fragment defined in (iii), wherein the amino acid sequence has at least 10 amino acids, and less than 805 amino acids.

In particular, the inserted nucleic acid may comprise a nucleotide sequence comprising:
(i) the nucleotide sequence of SEQ ID NO: 2 or SEQ ID NO: 4,
(ii) a nucleotide sequence that shares at least 70 percent identity with the nucleotide sequence of SEQ ID NO: 2 or SEQ ID NO: 4,
(iii) a fragment of the nucleotide sequence of SEQ ID NO: 2 or SEQ ID NO: 4, which fragment has at least 30 contiguous nucleotides of the nucleotide sequence of SEQ ID NO: 2 or SEQ ID NO: 4,
(iv) a nucleotide sequence sharing at least 70 percent identity with the fragment defined in (iii), wherein the nucleotide sequence has at least 30 nucleotides and wherein the nucleotide sequence has less than 2018 nucleotides,
(v) a nucleotide sequence that hybridises with the nucleotide sequence of SEQ ID NO: 2 or SEQ ID NO: 4 under stringent conditions, or
(vi) a nucleotide sequence that encodes a polypeptide as defined in this specification.

Preferably, the nucleotide sequence encodes an agent that inhibits the ability of a leptin to activate the Ob-Rb receptor and/or binding of leptin to the Ob-Rb receptor.

The transgenic non-human animal preferably expresses the inserted nucleic acid.

As the Ob-Rb receptor is a neuronal cell transmembrane receptor, it is believed that expressing the agent in close proximity to the Ob-Rb receptor will allow the agent to bind to any leptin in the vicinity of the receptor before the leptin binds to the receptor. This provides for efficient inhibition of the Ob-Rb receptor mediated leptin signaling pathway. Also, this has the advantage that expression of the agent should not significantly interfere with leptin mediated signaling in cells other than neuronal cells, i.e. in non-nervous system cells.

Preferably, the animal expresses the agent specifically in the nervous system, e.g. in neuronal cells, and is preferably not expressed in non-nervous system tissue. This may be achieved by operably linking the nucleotide sequence encoding the agent to a regulatory element, e.g. a promoter nucleic acid, from a protein or polypeptide that is normally expressed in the nervous system of the animal and that is preferably not expressed in non-nervous system tissues. A nucleic acid that is specifically expressed in the nervous system means, for example, that the nucleic acid is substantially or predominantly only expressed in the nervous system of the animal, e.g. it is not expressed in cells other than nervous system cells. Likewise, a nucleic acid that is specifically expressed in neuronal cells means, for example, that the nucleic acid is substantially or predominantly only expressed in neuronal cells, e.g. it is not expressed in cells other than neuronal cells.

The transgenic non-human animal may be any non-human animal. However, the animal is preferably a mammal, more preferably a rodent, e.g. a mouse or a rat, most preferably a mouse. The transgenic non-human animal preferably has increased susceptibility for obesity and/or neurodegenerative disease, e.g. compared to the wild type animal. For example, animals expressing the agent may be statistically more likely, e.g. with a statistical significance of P<0.05, to develop obesity and/or neurodegenerative disease. Preferably, the transgenic non-human animal is a model for obesity, and/or neurodegenerative disease. The neurodegenerative disease may be Alzheimer's disease.

In a further aspect, there is provided a transgenic non-human animal having a nucleic acid inserted in its genome, wherein the nucleic acid comprises a nucleotide sequence encoding Ob-Re, wherein the Ob-Re encoded by the nucleotide sequence is specifically expressed in the nervous system, e.g. in neuronal cells.

In a further aspect, there is provided a transgenic non-human animal having a nucleic acid inserted in its genome, wherein the nucleic acid comprises a heterologous construct, which construct comprises a nucleotide sequence encoding Ob-Re operably linked to a regulatory element, wherein the Ob-Re encoded by the nucleotide sequence is specifically expressed in the nervous system, e.g. in neuronal cells. In a further aspect, there is provided a transgenic mouse having a nucleic acid inserted in its genome, wherein the nucleic acid comprises a heterologous construct, which construct comprises a nucleotide sequence encoding Ob-Re operably linked to a regulatory element, wherein the Ob-Re encoded by the nucleotide sequence is specifically expressed in the nervous system, e.g. in neuronal cells.

In a further aspect of the invention, there is provided a method of producing a transgenic non-human animal, comprising the steps of:

(i) inserting a nucleic acid into the genome of a non-human animal germ line cell; and
(ii) generating a non-human animal from said non-human animal germ line cell;

wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor.

In a further aspect, there is provided a method of identifying and/or obtaining a compound useful in the treatment of obesity, comprising the steps of:

(i) administering a test compound to a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor; and
(ii) determining the effect of the test compound on the susceptibility of the animal to gain weight.

Preferably, a reduction in the susceptibility of the non-human animal to gain weight in the presence of the compound relative to the absence of the test compound is indicative that the compound is useful in the treatment of obesity. Preferably, the method comprises the step of identifying a test compound as useful in the treatment of obesity.

In a further aspect, there is provided a method of identifying and/or obtaining a compound that represses the development of obesity, comprising the steps of:

(i) providing a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor, and wherein the animal has a capacity to gain weight;
(ii) administering a test compound to the animal, and
(iii) monitoring the weight gain in the animal.

An animal that has a capacity to gain weight is preferably an animal that has a capacity to become obese, e.g. an animal that has not developed complete obesity. An animal that is considered obese is preferably an animal, e.g. an adult animal, that has a body weight of which at least 25, 30, 35, 40, 45, 50, 55, or even 60 percent is due to fat tissue. Preferably, the animal is a mouse. A typical normal adult mouse has body fat of about 10-20 percent by weight.

Preferably, a lower weight gain, e.g. a significantly lower weight gain, in the non-human animal administered with the test compound relative to the weight gain in the transgenic non-human animal not administered with the test compound is indicative that the test compound represses the development of obesity. For example, a reduction in the rate of weight increase in the animal relative to the animal not administered with the test compound is indicative that the test compound represses the development of obesity. Preferably the animal gains weight at a rate that is 10, 20, 30, 40, 50, 60, 70, 90, or 100 percent slower than the rate at which the animal not administered with the test compound gains weight. Preferably, the method comprises the step of identifying the test compound as useful in the treatment of obesity.

In a further aspect, there is provided a method of identifying and/or obtaining a compound useful in the treatment of neurodegenerative disease, comprising the steps of:

(i) administering a test compound to a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor; and
(ii) determining the effect of the test compound on neurodegeneration.

Preferably, a reduction, e.g. a significant reduction, in neurodegeneration in said non-human animal in the presence of the test compound relative to the absence of the test compound is indicative that the compound is useful in the treatment of neurodegenerative disease. Preferably, the method comprises the step of identifying the compound as useful in the treatment of neurodegenerative disease.

In a further aspect, there is provided a method of identifying and/or obtaining a compound that represses the development of neurodegenerative disease, comprising the steps of:

(i) providing a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor, and wherein the animal has a capacity for neurodegeneration;
(ii) administering a test compound to the animal, and
(iii) monitoring the development of neurodegeneration in the animal.

An animal that has a capacity for neurodegeneration may be an animal has intact brain tissue that may undergo neurodegeneration. Preferably the animal has no neurodegeneration and/or does not have neurodegenerative disease at commencement of the study.

The development of neurodegeneration may be measured and/or determined by using behaviour tests, such as cognitive tests. Examples of cognitive tests include various forms of maze tests, e.g. water maze.

Alternatively, development of neurodegeneration may be measured and/or determined by monitoring the accumulation of amyloid plaque formation and/or accumulation of Aβ in the brain of the animal. Other tests may include synaptic activity monitoring using electrophysiology or optical imaging methods. It is generally accepted that neurodegeneration starts with defects in synaptic activity and/or morphology.

Aβ peptide is the major proteinateuos component of the amyloid plaques found in the brains of Alzheimer's disease patients. It has been reported that the amount of extracellular accrual of Aβ is critical for the pathobiology of Alzheimer's disease (Fewlass et al.).

Preferably, a reduction in the development of neurodegeneration in the animal administered with the test compound relative to the development of neurodegeneration in the animal not administered with the test compound is indicative that the compound represses the development of neurodegenerative disease. For example, a reduction in the development of neurodegeneration means that the animal undergoes neurodegeneration at a slower rate, and/or does not undergo any neurodegeneration compared to negative control. Preferably the animal administered with the test compound undergoes neurodegeneration at a rate that is 10, 20, 30, 40, 50, 60, 70, 90, or 100 percent slower than the rate at which the animal not administered with the test compound undergoes neurodegeneration. Preferably, the method comprises the step of identifying the compound as useful in the treatment of neurodegenerative disease.

In a further aspect, there is provided use of a transgenic non-human animal, as defined herein, in testing, e.g. identifying, compounds for their ability to repress the development of obesity or neurodegenerative disease. For example, use of an animal in testing compounds that are capable of repressing the development of obesity or neurodegenerative disease.

Preferably the neurodegenerative disease is Alzheimer's disease.

In a further aspect, the invention provides compounds, which compounds have been identified by methods described in this specification as useful for treating obesity, repressing the development of obesity, useful for treating neurodegenerative disease, and/or repressing the development of neurodegenerative disease.

In a further aspect, there is provided use of a transgenic non-human animal, as defined in this specification, in testing compounds for their ability to repress the development of obesity and/or neurodegenerative disease, e.g. Alzheimer's disease.

In any method of the invention, the inserted nucleic acid may be any nucleic acid as defined in this specification, the agent may be any agent as defined in this specification, and the transgenic non-human animal may be any transgenic non-human animal as defined in this specification.

In particular, in any method of the invention, the transgenic non-human animal may additionally or alternatively be a non-human animal that has a nucleic acid inserted in its genome, wherein the presence of the inserted nucleic acid in the genome of the animal results in expression of an agent, which agent is encoded by a nucleotide sequence in the genome of the animal, wherein the agent inhibits the ability of a leptin to activate an Ob-Rb receptor.

### BRIEF DESCRIPTION OF THE FIGURES

Embodiments and experiments illustrating the principles of the invention will now be discussed with reference to the accompanying figures in which:

FIG. 1

FIG. 1 shows a schematic diagram outlining how interference with leptin signaling by Ob-Re may lead to obesity and Alzheimer's disease. The diagram indicates that loss of leptin signaling may lead to an increase in β-secretase activity and a reduction in Apo-E-dependent Aβ uptake. This may lead to an increase in Aβ accumulation and Alzheimer's disease. In addition, an increase in β-secretase activity may lead to an increase γ-secretase activity which in turn may lead to an increase in Ob-Re production, thereby creating a positive feedback signaling mechanism.

FIG. 2

FIG. 2 shows the amino acid sequence (SEQ ID NO: 1) and nucleotide sequence (SEQ ID NO: 2) of Ob-Re. The sequences were obtained from GenBank under accession number U49110.1 (GI: 1195492). FIG. 2 also shows the amino acid sequence (SEQ ID NO: 3) of the leptin binding domain of Ob-Re. This corresponds to amino acids 323-640 of SEQ ID NO: 1, not including the N-terminal methionine. SEQ ID NO: 4 shows the corresponding coding nucleotide sequence of SEQ ID NO: 3.

FIG. 3

FIG. 3 shows the nucleic acid sequence (SEQ ID NO: 5) of the vector pThy1-wp1, which has 9163 bases. This vector includes the Thy-1 promoter sequence and was used to construct the transgenic mouse model as described in the Examples.

FIG. 4

FIG. 4 shows the amino acid sequence (SEQ ID NO: 6) and coding nucleotide sequence (SEQ ID NO: 7) of mouse leptin. The sequences were obtained from GenBank under accession number U18812.1 (GI:746416).

FIG. 5

FIG. 5 shows the amino acid sequence (SEQ ID NO: 8) and coding nucleotide sequence (SEQ ID NO: 9) of mouse Ob-Rb. The sequences were obtained from GenBank under accession number U46135.1 (GI:1184113).

FIG. 6

FIG. 6 shows a nucleotide sequence (SEQ ID NO: 10) that includes the Thy-1 promoter nucleotide sequence. This sequence corresponds to nucleotides 999-3145 of pThy1-wp1.

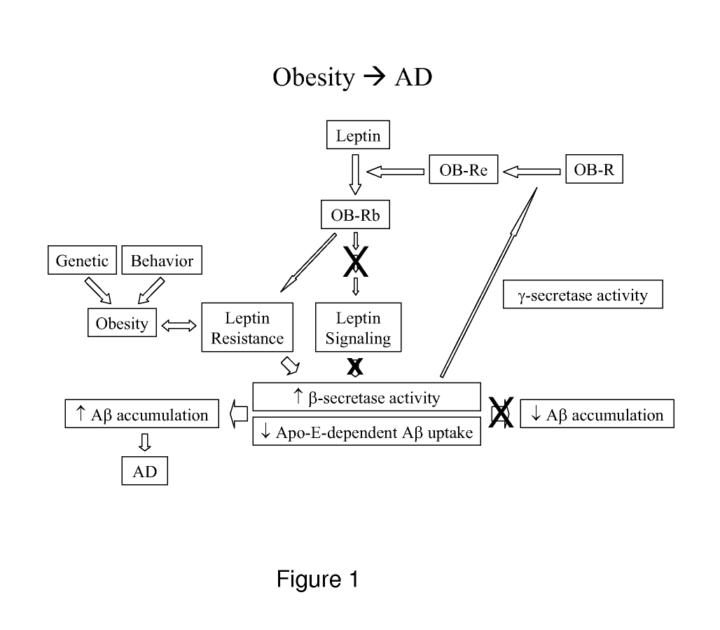

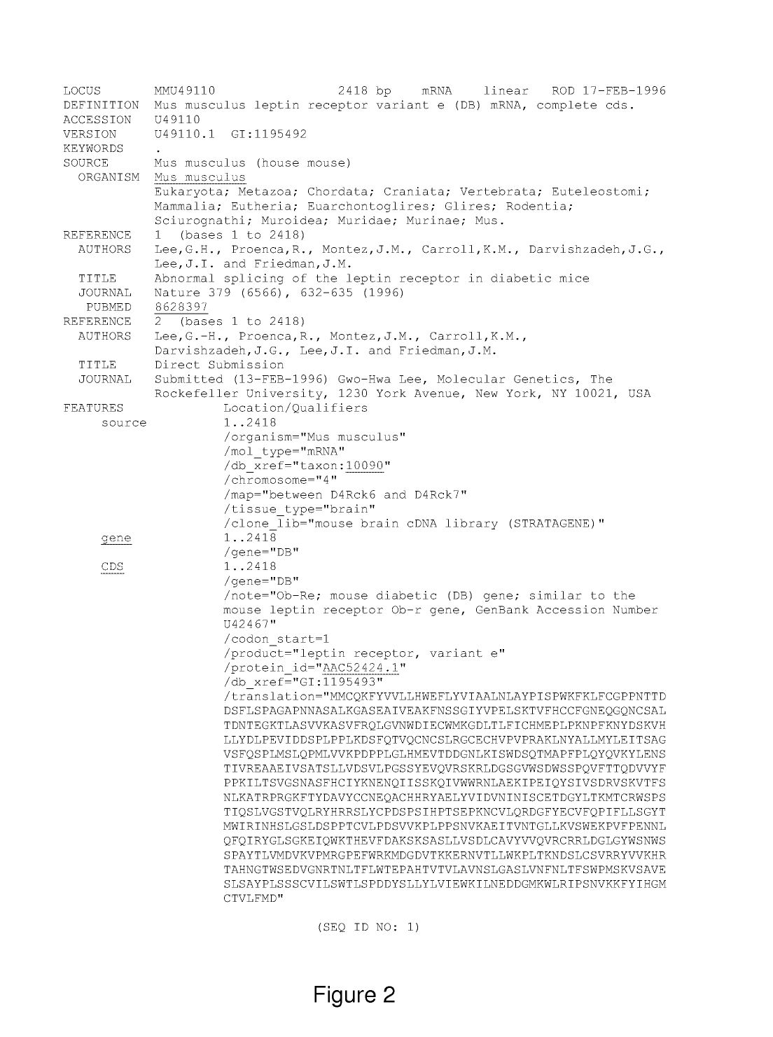

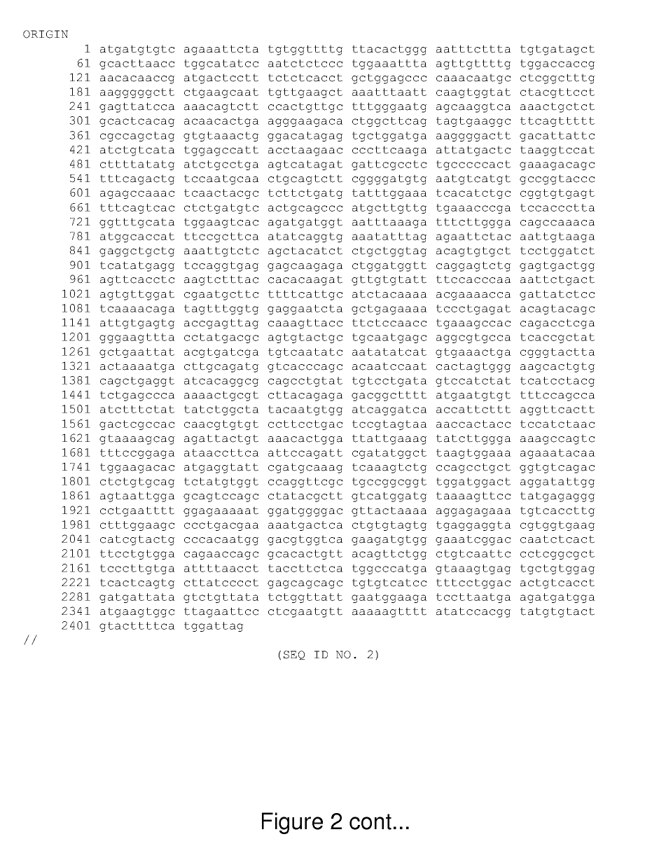

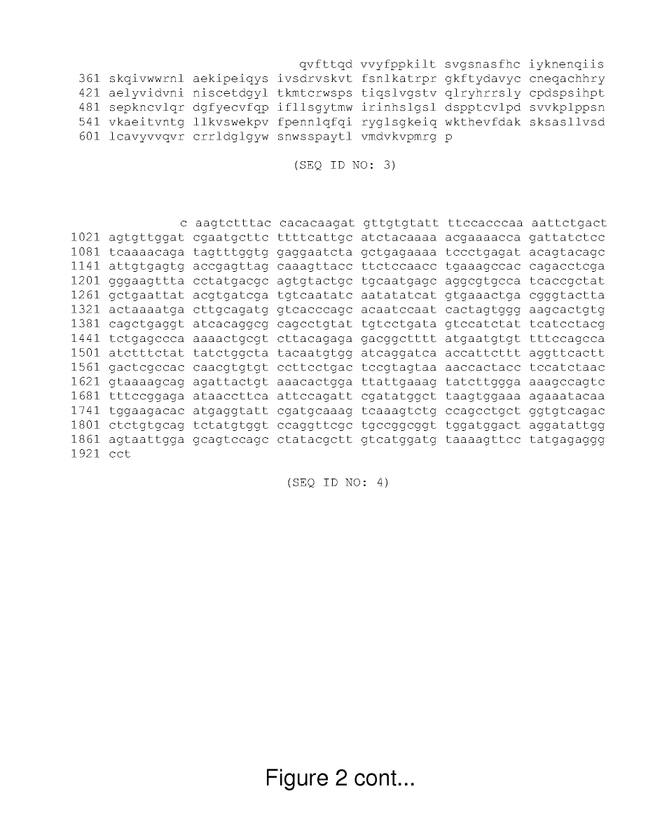

### DETAILED DESCRIPTION OF THE INVENTION

### The Leptin Signaling Pathway

Leptin binding to the long form of the leptin receptors Ob-Rb initiates a signaling cascade, which originates from three major sites within the activated receptor complex: two from the Ob-Rb itself (Tyr985 and Tyr1138) and one from Ob-Rb associated Jak2 molecule. Activation of Tyr985 on Ob-Rb recruits the binding of the protein tyrosine phosphatase SHP-2, which mediates p21 ras-ERK pathway; phosphorylation of Tyr1138 specifically binds and activates the latent transcription factor, signal transducer and activator of transcription-3 (STAT3); and phosphorylation of Jak2 activates IRS-PI3K pathway. Tyr1138-STAT3 pathway is required for leptin regulation of energy balance. Mice with neural deletion of STAT3 exhibit severe obesity and diabetes. (Gao et al. 2004).

Normal leptin signaling has also been linked with reduced β-secretase activity and enhanced ApoE-dependent Aβ-clearance. The combined effects are reduced accumulation of Aβ, the major component of Amyloid plaque. Amyloid plaque is tightly associated with and may be the cause of Alzheimer's disease (AD). Therefore, interference of leptin signaling could remove the normal neuronal protection mechanism by leptin, and lead to increased accumulation of Aβ, thereby increasing the probability of Alzheimer's disease.

Disruption of leptin signaling, or leptin resistance may be achieved by blocking the pathways described earlier (ERK, PI3K and STAT3), or by preventing circulating leptin from accessing brain targets. SLR has been shown to bind tightly with Leptin in the plasma. This sequestration action may reduce leptin from binding and activating Ob-Rb, and therefore impair normal leptin signaling and increase β-secretase activity.

SLR may be generated by alternative splicing of OBR gene, or by regulated intramembrane proteolysis (RIP) and ectodomain shedding from the other leptin receptors. Initial cleavage of transmembrane proteins by β-secretase generates a short extracellular fragment. This may trigger a second cleavage event by γ-secretase and release the remaining ectodomain. Sequential proteolysis of transmembrane-containing OBRs triggered by increased β- and γ-secretase activities may produce and release excessive SLR into circulation. The increased level of SLR, in turn chelates and further reduce the availability of Leptin from acting on OB-Rb, and causes leptin resistance, which would further enhance the production of SLR. Thus, these steps form a positive feedback loop, a vicious cycle that leads to leptin resistance and enhanced accumulation of Aβ.

Leptin resistance in animals has been shown to lead to severe obesity, and diet-induced obesity animals have also been shown to be resistant to leptin. The cause of obesity is complex, involving both genetic and environmental factors. Through leptin signaling and its regulation of Aβ accumulation, obesity may be linked to AD. Since SLR chelation of leptin may result in leptin resistance, SLR could be a link between obesity and AD.

To understand the biological and pathological basis of obesity and neurodegeneration, such as AD, one can tip the balance of the feedback system described above to generate pathological changes. To generate stable and renewable mouse models, the inventor decided to take a genetic approach by over-expression of SLR in the brain. In this mouse model, normal leptin signaling is interrupted specifically in the central nervous system, without affecting leptin's peripheral actions in the adipose tissue and pancreas, etc. The inventor expects the mouse model to exhibit impaired leptin signaling, increased body weight, and Aβ accumulation and neurodegeneration, which eventually leads to AD.

The animal model described here allows verification in an in vivo system of the importance of leptin signaling in neurodegeneration. First, molecules involved in the leptin signaling pathway can be tested for their effects in reducing obesity. Second, the compounds or molecules that enhance leptin signaling can then be tested on the mouse model for their effects on neuroprotection.

### Leptin

The term “leptin” may refer to any leptin molecule produced by any animal, including human and non-human animals. Preferably, the term refers to leptin produced by rodents, in particular leptin produced by mouse. Preferably, the term refers to leptin encoded by the nucleic acid sequence shown in SEQ ID NO: 7. A leptin may comprise, consist of, or consist essentially of, the amino acid sequence shown in SEQ ID NO: 6.

Recombinant leptin is readily commercially available from companies such as Sigma® and Invitrogen®. Alternatively, leptin may be produced in mammalian cells or bacterial cells by transfecting or transforming the respective cells with a DNA construct containing leptin coding sequence under an appropriate promoter.

### Ob-Rb Receptor

The term “Ob-Rb receptor” refers to the long isoform of the Ob receptor (Ahim et al.). The term may refer to any Ob-Rb receptor produced by any animal, including human and non-human animals. Preferably, the term refers to Ob-Rb receptor produced by rodents, in particular Ob-Rb receptor produced by mouse. Preferably, the term refers to Ob-Rb encoded by the nucleic acid sequence shown in SEQ ID NO: 9. An Ob-Rb receptor may comprise, or consist of, the amino acid sequence shown in SEQ ID NO: 8.

### Ob-Re

The term “Ob-Re receptor” refers to the soluble form of the Ob receptor (Ahim et al.). The term “Ob-Re” may refer to any Ob-Re produced by any animal, including human and non-human animals. Preferably, the term refers to Ob-Re receptor produced by rodents, in particular Ob-Re receptor produced by mouse. Preferably, the term refers to Ob-Re encoded by the nucleic acid sequence shown in SEQ ID NO: 2. An Ob-Re receptor may comprise, or consist of, the amino acid sequence shown in SEQ ID NO: 1.

### Agents

The agent may be a peptide, polypeptide or protein. For example, the nucleic acid may encode a polypeptide that inhibits the ability of a leptin to activate the Ob-Rb receptor and/or an agent that inhibits binding of a leptin to an Ob-Rb receptor. Preferably, where the agent inhibits binding of a leptin to an Ob-Rb, the leptin and Ob-Rb are derived from the same species, more preferably the same organism.

Preferably, the agent inhibits the ability of a leptin to activate an Ob-Rb receptor. Where the ability of a leptin to activate an Ob-Rb receptor is determined by using luciferase assay system, this may be achieved by cloning the luciferase gene downstream of a promoter that is regulated by one of the leptin-activated signaling molecules. The inventor has designed an in vitro system that is based on this principle. There are two key components in this system: first, a stable HEK293 cell line was generated to over-express the long form of leptin receptor (OB-Rb); second, luc gene was cloned downstream of the phosphor-STAT3 binding/regulatory DNA sequence (the construct was named m67-luc). When m67-luc is expressed in the above-mentioned stable cell line, upon leptin stimulation, luciferase gene would be turned on and expressed. The luciferase gene activity can be conveniently measured using standard luciferase assays. This system can be used to test whether any agents can prevent leptin from activating its receptors through standard commercially available luciferase assays.

Preferably, the agent inhibits the ability of a leptin to activate an Ob-Rb receptor by at least 10, 20, 30, 40, 50, 60, 70, 80, 90, 95, 96, 97, 98, 99, or even 100 percent compared to activation of the Ob-Rb receptor in the absence of agent.

Preferably, the agent specifically binds to a leptin, e.g. it selectively binds to a leptin and/or binds to leptin with high affinity. For example the agent may bind a leptin with a Kd of less than, or more than, 500, 400, 300, 200, 100, 90, 80, 70, 60, 50, 40, 30, 20, or 10 μM, for example less than, or more than, 1000, 500, 400, 300, 200, 100, 90, 80, 70, 60, 50, 40, 30, 20, or 10 μM. For example, the agent may bind a leptin with a Kd of 1000 μM to 100 μM, 100 μM to 100 nM, 10 μM to 100 nM, 1 μM to 100 nM. For example, the agent may bind leptin with a Kd of 1000 μM to 10 nM, 100 μM to 10 nM, 10 μM to 10 nM, 1 μM to 10 nM or 100 M to 10 nM. For example, the agent may bind leptin with a Kd of 100 μM to 1 nM, 100 μM to 1 nM, 100 μM to 1 nM, or 100 mM to 1 nM. Methods of determining binding affinity are well-known in the art, e.g. through biding assays or by using Surface Plasmon Resonance. Preferably, the ability of the agent to bind a leptin is measured in a solution that mimics the physiological conditions.

The Ob-Re isoform of the Ob-Rb receptor comprises the leptin binding domain of the Ob-Rb receptor. Thus, an agent that inhibits and/or prevents a leptin from binding to the Ob-Re receptor may also inhibit leptin from binding to Ob-Rb to a similar extent. Thus, the ability of leptin to bind Ob-Rb may be assessed by measuring the ability of leptin to bind Ob-Re. The ability to inhibit and/or prevent leptin binding to Ob-Re may be determined by binding Ob-Re to a solid support and measuring binding of leptin to Ob-Re in the presence of the agent and in the absence of the agent, e.g. using surface Plasmon resonance. Such methods are well known to the person skilled in the art.

By binding to leptin, the agent preferably chelates leptin. For example, the bound leptin is thereby made unavailable for binding to the Ob-Rb receptor with the effect of reducing, repressing or inhibiting leptin mediated signaling and inducing full or partial leptin resistance.

The agent may comprise an amino sequence that shares at least 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent sequence identity with the amino acid sequence of SEQ ID NO: 1. The sequence identity may be shared over 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or 805 contiguous amino acids of the sequence of SEQ ID NO: 1. Preferably, the sequence identity is shared over the full-length sequence of SEQ ID NO: 1.

The agent may comprise a fragment of the amino acid sequence of SEQ ID NO: 1, which fragment may have less than, or at least, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or 804 contiguous amino acids of the amino acid sequence of SEQ ID NO: 1.

The agent may comprise an amino acid sequence that shares at least 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent sequence identity with a fragment of the amino acid sequence of SEQ ID NO: 1. An amino acid sequence sharing at least a particular sequence identity with a fragment of the amino acid sequence of SEQ ID NO: 1 may comprise less than, or at least, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, or 805 contiguous amino acids.

### Nucleic Acids

An heterologous nucleic acid is preferably a nucleic acid that is outside its natural environment, e.g. it is not naturally occurring within the non-human animal. The heterologous nucleic acid may, for example, have been introduced into the non-human animal or an ancestor of the non-human animal by recombinant techniques (e.g. it may be a recombinant nucleic acid). The inserted nucleic acid may comprise, consist of, or consist essentially of, heterologous nucleic acid.

A construct is preferably a nucleic acid comprising a coding nucleic acid sequence operably linked to a regulatory element, e.g. a promoter. A heterologous construct is preferably heterologous in the sense that the coding sequence is not operably linked to the regulatory sequence of the heterologous construct in the genome of the wild-type animal. However, either or both of the regulatory element and nucleic acid sequence may be endogenous to the non-human animal.

A nucleotide sequence that encodes the agent may share a sequence identity of at least 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 percent sequence identity with the nucleotide sequence of SEQ ID NO: 2. The sequence identity may be shared over 30, 60, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, 1110, 1140, 1170, 1200, 1230, 1260, 1290, 1320, 1350, 1380, 1410, 1440, 1470, 1500, 1530, 1560, 1590, 1620, 1650, 1680, 1710, 1740, 1770, 1800, 1830, 1860, 1890, 1920, 1950, 1980, 2010, 2040, 2070, 2100, 2130, 2160, 2190, 2220, 2250, 2280, 2310, 2340, 2370, 2400, or 2418 contiguous nucleotides of SEQ ID NO: 2. Preferably, the sequence identity is shared over the full-length sequence of SEQ ID NO: 2.

A fragment of the nucleotide sequence of SEQ ID NO: 2 may have less than, or at least, 30, 60, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, 1110, 1140, 1170, 1200, 1230, 1260, 1290, 1320, 1350, 1380, 1410, 1440, 1470, 1500, 1530, 1560, 1590, 1620, 1650, 1680, 1710, 1740, 1770, 1800, 1830, 1860, 1890, 1920, 1950, 1980, 2010, 2040, 2070, 2100, 2130, 2160, 2190, 2220, 2250, 2280, 2310, 2340, 2370, 2400, or 2418 contiguous nucleotides of the nucleic acid sequence of SEQ ID NO: 2.

A nucleotide sequence sharing at least a particular sequence identity with a fragment of the nucleotide sequence of SEQ ID NO: 1 may share at least 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent sequence identity with the fragment. The sequence identity is shared over the full-length sequence of the fragment.

A nucleotide sequence sharing at least a particular sequence identity with a fragment of the nucleotide sequence of SEQ ID NO: 1, may have less than, or at least, 30, 60, 90, 120, 150, 180, 210, 240, 270, 300, 330, 360, 390, 420, 450, 480, 510, 540, 570, 600, 630, 660, 690, 720, 750, 780, 810, 840, 870, 900, 930, 960, 990, 1020, 1050, 1080, 1110, 1140, 1170, 1200, 1230, 1260, 1290, 1320, 1350, 1380, 1410, 1440, 1470, 1500, 1530, 1560, 1590, 1620, 1650, 1680, 1710, 1740, 1770, 1800, 1830, 1860, 1890, 1920, 1950, 1980, 2010, 2040, 2070, 2100, 2130, 2160, 2190, 2220, 2250, 2280, 2310, 2340, 2370, 2400, or 2418 contiguous nucleotides of the nucleic acid sequence of SEQ ID NO: 2.

The nucleotide sequence encoding the agent may be linked, e.g. operably linked, to a regulatory element, in particular a neuronal specific regulatory element, e.g. a promoter, such as Thy-1 (e.g. SEQ ID NO: 10). Thus the nucleic acid encoding the agent may be operably linked to a promoter nucleic acid from a protein or polypeptide that is normally expressed in the nervous system of the animal and that is preferably not expressed in non-nervous system tissues, i.e. it is normally substantially or predominantly only expressed in the nervous system of the animal.

In a further aspect, there is provided a nucleic acid comprising a nucleotide sequence encoding an agent, which agent is one defined herein, e.g. Ob-Re, operably linked to a nucleotide sequence encoding a neuron-specific promoter, e.g. Thy-1 (e.g. SEQ ID NO: 10).

In this specification the term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence. Where appropriate, the resulting transcript may then be translated into a desired protein or polypeptide.

Reference in this specification to nucleic acid preferably refers to DNA.

### Methods of Producing Transgenic Non-Human Animals

The nucleotide sequence encoding the agent may include a regulatory element and may be comprised in a vector for introduction into a non-human animal.

A suitable non-human animal germ-line cell may include an egg, oocyte or embryonic stem (ES) cell. In some embodiments, the nucleic acid may be introduced into a germ line cell that is comprised in an early stage embryo, such as a blastocyst.

The nucleic acid or vector may be introduced into the germ line cell using any method known in the art, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyornithine, etc., (See, for example Van der Putten, et al., 1985, Proc. Natl. Acad. Sci., USA 82:6148-6152; Thompson, et al., 1989, Cell 56:313-321; Lo, 1983, MoI Cell. Biol. 3:1803-1814; Lavitrano, et al., 1989, Cell, 57:717-723 Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

After the nucleic acid or vector has been introduced into cells, the cells in which the nucleic acid has successfully incorporated into the non-human animal germ-line cell genome may be identified.

Successful insertion of the nucleic acid into the genome may be confirmed by analyzing the DNA of the selected cells using routine techniques, such as PCR and/or Southern analysis.

A non-human animal may be generated from a cell comprising the inserted nucleic acid or vector using standard techniques (see for example Piedrahita et al (1992) PNAS USA 89 4471-4475, Roller et al (1989) PNAS USA 86 8927-8931; Transgenic Animal Technology: A Laboratory Handbook, Pinkert C A (2002) Academic Press).

For example, the cell may be introduced into a blastocyst. The injection of transformed cells injected into a non-human animal blastocyst may lead to the formation of chimeras (see e.g., Bradley, A. in Teratocarcinomas and Embryonic Stem Cells: A Practical Approach, E. J. Robertson, ed., IRL, Oxford, pp. 113-152 (1987)). Alternatively, germ-line cells identified as comprising the inserted nucleic acid may be allowed to aggregate with dissociated mouse embryo cells to form an aggregation chimera. A chimeric embryo can then be implanted into a suitable pseudopregnant female foster non-human animal and the embryo brought to term. Chimeric progeny harboring the inserted nucleic acid in their germ cells can be used to breed non-human animals in which all cells of the non-human animal comprise the inserted nucleic acid.

Non-human animals produced as described may be crossed with non-human animals of the same or other genotypes to produce descendents. The genotype of the non-human animal or descendent may be determined. A method may include determining that the non-human animal or the descendent specifically expresses the nucleotide sequence encoding the agent. Methods of determining the genotype of a non-human animal are well-known in the art.

### Methods of Identifying Compounds

The test compound may be administered to the non-human animal by any convenient method, for example injection, including intravenous, cutaneous, subcutaneous or intraperitoneal injection. A test compound suitable for use in a method described herein may be any compound or molecular entity, such as a small organic molecule, peptide or nucleic acid.

A method described herein may comprise identifying a test compound as useful in the treatment of obesity and/or neurodegenerative disease, for example Alzheimer's disease. Following identification of a compound using a method described above, the compound may be isolated and/or synthesized.

The identified compound may be synthesized using conventional chemical synthesis methodologies. Methods for the development and optimization of synthetic routes are well known to persons skilled in this field.

A compound identified using a method described herein may be assessed or investigated further using one or more secondary screens. For example the toxicology and/or biological effect of the compound may be determined in wild-type non-human animals.

Following performance of a method described herein, the non-human animal may be sacrificed or euthanized.

The compound may be modified to optimize its pharmaceutical properties. The modified compound may be tested using the methods described herein to see whether it has the target property, or to what extent the target property is exhibited. Modified compounds include mimetics of the lead compound. Further optimization or modification can then be carried out to arrive at one or more final compounds for in vivo or clinical testing.

In a further aspect, there is provided a compound identified by a method described herein, which compound is useful for treating obesity and/or neurodegenerative disease, e.g. Alzheimer's disease.

### Test Compounds

A test compound may modulate or interfere with the interaction of molecules, e.g. of two proteins, in one of a number of ways. In one arrangement the compound may directly modulate the interaction by binding to one molecule, masking the site of interaction. Candidate compounds may comprise small molecule, synthetic or naturally occurring, specific inhibitors and may be enzyme active site inhibitors, either competitive or non-competitive. Alternatively, a test compound may comprise a peptide which interacts with the target molecule or an organic compound mimicking the peptide structure.

Preferred test compounds include PTP1B inhibitors, SOCS3 inhibitors, and PTEN inhibitors.

PTP1B Inhibitors

PTP1B is a negative regulator of OBRb-Jak2. Inhibition of PTP1B enhances leptin signaling. Compounds such as benzofuran, benzothiophene biphenyl, and vanadate, have been shown to be potent inhibitors of PTP1B.

### SOCS3 Inhibitors

SOCS3 is a negative regulator of STAT3, a key signaling molecule downstream of leptin-leptin receptor activation. Inhibition of SOCS3 increases the level/activity of STAT3, and in turn enhances leptin signaling. SOCS3 activity may be inhibited by specific antibodies raised against it, or by a compound/molecule that prevents its phosphorylation.

### PTEN Inhibitors

PTEN promotes PIP3 to PIP2, which indirectly inhibits PI3K and AKt activation, a key leptin signaling pathway. Inhibitors of PTEN therefore increase PIP3, and subsequent PI3K/AKt activity, and thus enhance leptin signaling. Some inhibitors are commercially available, for example, Dipotassium Bisperoxo(5-hydroxypyridine-2-carboxyl)oxovanadate and Potassium Bisperoxo(1,10-phenanthroline)oxovanadate from Calbiochem.

These compounds may be provided for treating neurodegenerative disease, e.g. Alzheimer's disease, and/or obesity. In particular, these compounds may be identified by methods described in this specification as useful for treating obesity, repressing the development of obesity, useful for treating neurodegenerative disease, and/or repressing the development of neurodegenerative disease.

Other examples of candidate test compounds include non-functional homologues of the target molecule, antibodies and antibody products, e.g. monoclonal and polyclonal antibodies, single chain antibodies, chimeric antibodies and CDR-grated antibodies, which recognize one of the interacting molecules, a complex of bound molecules, or a domain or region of a protein which is involved in the interaction.

### Pharmaceutical Preparations

The test compound may be used in the preparation, i.e. manufacture or formulation, of a composition such as a medicament, pharmaceutical composition or drug. These may be administered to individuals, e.g. for the treatment of a condition described herein. A method may comprise formulating the test compound or the modified test compound in a pharmaceutical composition with a pharmaceutically acceptable excipient, vehicle and/or carrier. Suitable acceptable excipients, vehicles and carriers are well-known in the art.

In accordance with the present invention methods are also provided for the production of pharmaceutically useful compositions, which may be based on a substance or test compound so identified. In addition to the steps of the methods described herein, such methods of production may further comprise one or more steps selected from:
(a) identifying and/or characterizing the structure of a selected substance or test compound;
(b) obtaining the substance or compound;
(c) mixing the selected substance or compound with a pharmaceutically acceptable carrier, adjuvant or diluent.

For example, a further aspect of the present invention relates to a method of formulating or producing a pharmaceutical composition for use in the treatment of obesity and/or neurodegeneration, the method comprising identifying a compound or substance in accordance with one or more of the methods described herein, and further comprising one or more of the steps of:

(i) identifying the compound or substance; and/or
(ii) formulating a pharmaceutical composition by mixing the selected substance, or a prodrug thereof, with a pharmaceutically acceptable carrier, adjuvant or diluent.

The active agent may be present in the pharmaceutical composition so produced and may be present in the form of a physiologically acceptable salt.

Thus, in a further aspect, the invention provides a therapeutic composition effective in the prevention or treatment of obesity and/or neurodegenerative disease comprising a compound, as identified by a method as defined in this specification, and a pharmaceutically acceptable diluent, excipient and/or vehicle.

The term “pharmaceutically acceptable” as used herein pertains to compounds, materials, compositions, and/or dosage forms which are, within the scope of sound medical judgment, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

### Sequence Identity

Percentage (%) sequence identity is defined in this specification as the percentage of amino acid residues in a candidate sequence that are identical with residues in the given listed sequence (referred to by the SEQ ID No.) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

For example, where a given sequence comprises 100 amino acids and the candidate sequence comprises 10 amino acids, the candidate sequence can only have a maximum identity of 10% to the entire length of the given sequence. This is further illustrated in the following example:

(A)

| | | |
|---|---|---|
| Given seq: | XXXXXXXXXXXXXXX | (15 amino acids) |
| Comparison seq: | XXXXXYYYYYYY | (12 amino acids) |

The given sequence may be, for example, Ob-Re (e.g. SEQ ID NO:1).

% sequence identity=the number of identically matching amino acid residues after alignment divided by the total number of amino acid residues in the longer given sequence, i.e. (5 divided by 15)×100=33.3%

Where the comparison sequence is longer than the given sequence, sequence identity may be determined over the entire length of the given sequence. For example:

(B)

| | | |
|---|---|---|
| Given seq: | XXXXXXXXXX | (10 amino acids) |
| Comparison seq: | XXXXXYYYYYYZZYZZZZZZ | (20 amino acids) |

Again, the given sequence may be, for example, Ob-Re (e.g. SEQ ID NO:1).

% sequence identity=number of identical amino acids after alignment divided by total number of amino acid residues in the given sequence, i.e. (5 divided by 10)×100=50%.

Alignment for purposes of determining percent amino acid sequence identity can be achieved in various ways known to a person of skill in the art. Preferably, sequences are aligned using ClustalW 1.82 software, and using the default parameters, e.g. DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet, Protein/DNA ENDGAP=−1, and Protein/DNA GAPDIST=4. Other software that may be used includes BLAST, BLAST-2, ALIGN, ClustalW, T-coffee or Megalign (DNASTAR) software.

Identity of nucleic acid sequences may be determined in a similar manner involving aligning the sequences and introducing gaps if necessary, to achieve the maximum sequence identity, and calculating sequence identity over the entire length of the respective sequences. Where the aligned sequences are of different length, sequence identity may be determined as described above and illustrated in examples (A) and (B).

### Hybridisation Stringency

In accordance with the present invention, nucleic acid sequences may be identified by using hybridization and washing conditions of appropriate stringency.

Complementary nucleic acid sequences will hybridise to one another through Watson-Crick binding interactions. Sequences which are not 100% complementary may also hybridise but the strength of the hybridisation usually decreases with the decrease in complementarity. The strength of hybridisation can therefore be used to distinguish the degree of complementarity of sequences capable of binding to each other.

The “stringency” of a hybridization reaction can be readily determined by a person skilled in the art.

The stringency of a given reaction may depend upon factors such as probe length, washing temperature, and salt concentration. Higher temperatures are generally required for proper annealing of long probes, while shorter probes may be annealed at lower temperatures. The higher the degree of desired complementarity between the probe and hybridisable sequence, the higher the relative temperature which can be used. As a result, it follows that higher relative temperatures would tend to make the reaction conditions more stringent, while lower temperatures less so.

For example, hybridizations may be performed, according to the method of Sambrook et al., (“Molecular Cloning, A Laboratory Manual, Cold Spring Harbor Laboratory Press, 1989) using a hybridization solution comprising: 5×SSC, 5×Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2X SSC and 1% SDS; (2) 15 minutes at room temperature in 2X SSC and 0.1% SDS; (3) 30 minutes-1 hour at 37° C. in 1X SSC and 1% SDS; (4) 2 hours at 42-65° C. in 1X SSC and 1% SDS, changing the solution every 30 minutes.

One common formula for calculating the stringency conditions required to achieve hybridization between nucleic acid molecules is to calculate the melting temperature Tₘ (Sambrook et al., 1989):

Tₘ=81.5° C.+16.6 Log [Na+]+0.41(% G+C)−0.63(% formamide)−600/n

where n is the number of bases in the oligonucleotide.

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in sequence complementarity.

Accordingly, nucleotide sequences can be categorized by an ability to hybridise to a target sequence under different hybridisation and washing stringency conditions which can be selected by using the above equation. The Tₘ may be used to provide an indicator of the strength of the hybridisation.

The concept of distinguishing sequences based on the stringency of the conditions is well understood by the person skilled in the art and may be readily applied.

Sequences exhibiting 95-100% sequence complementarity are considered to hybridise under very high stringency conditions, sequences exhibiting 85-95% complementarity are considered to hybridise under high stringency conditions, sequences exhibiting 70-85% complementarity are considered to hybridise under intermediate stringency conditions, sequences exhibiting 60-70% complementarity are considered to hybridise under low stringency conditions and sequences exhibiting 50-60% complementarity are considered to hybridise under very low stringency conditions.

Various further aspects and embodiments of the present invention will be apparent to those skilled in the art in view of the present disclosure. All documents mentioned in this specification are incorporated herein by reference in their entirety.

The invention includes the combination of the aspects and preferred features described except where such a combination is clearly impermissible or expressly avoided.

The section headings used herein are for organizational purposes only and are not to be construed as limiting the subject matter described.

### EXAMPLES

Specific details of the best mode contemplated by the inventor for carrying out the invention are set forth below, by way of example. It will be apparent to one skilled in the art that the present invention may be practiced without limitation to these specific details.

### Example 1

### Transgenic Vector Construction

cDNA encoding the SLR (Ob-Re) was first amplified by PCR from genomic DNA extracted from the tail of a transgenic mouse over-expressing SLR in the liver. The following oligonucleotide pair was used for the PCR:

[SEQ ID No. 11]

| | |
|---|---|
| wh65 | (GCGAAGCTTATGATGTGTCAGAAATTCTATGTGG) |
| and | |

[SEQ ID No. 12]

| | |
|---|---|
| wh66 | (GCGGTCGACCTAATCCATGAAAAGTACAGTACAC). |

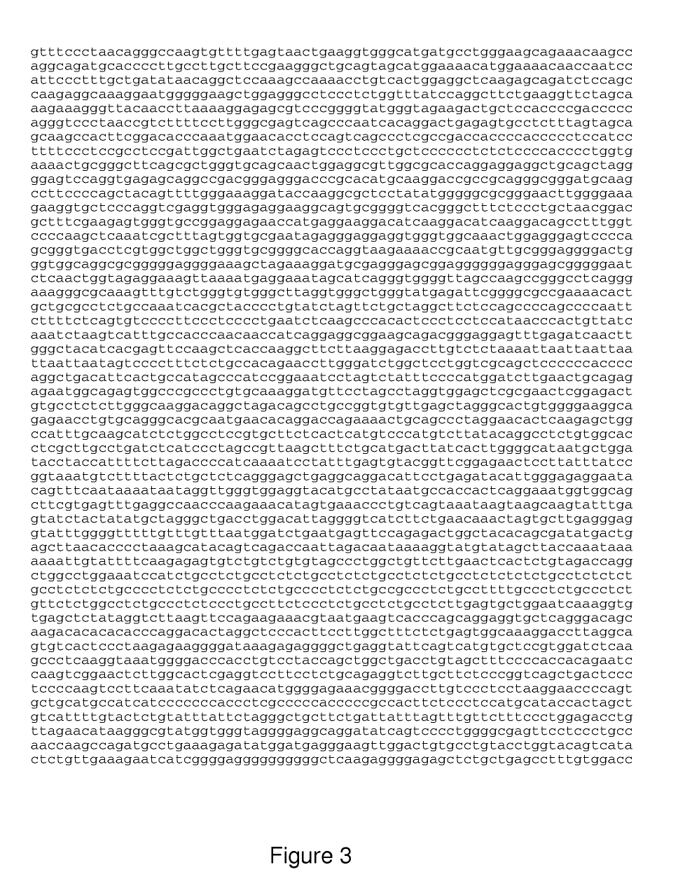

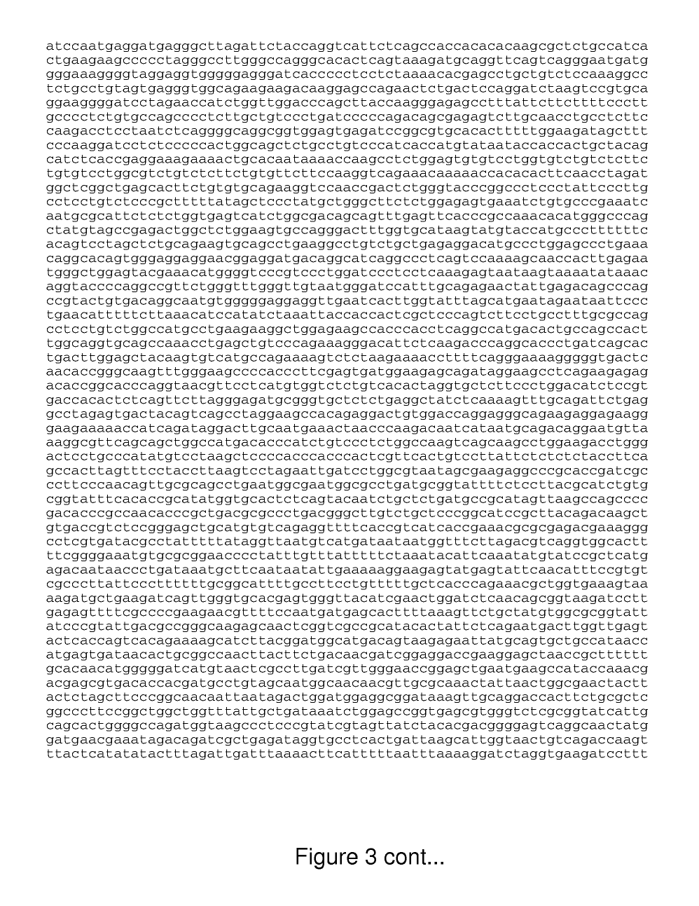

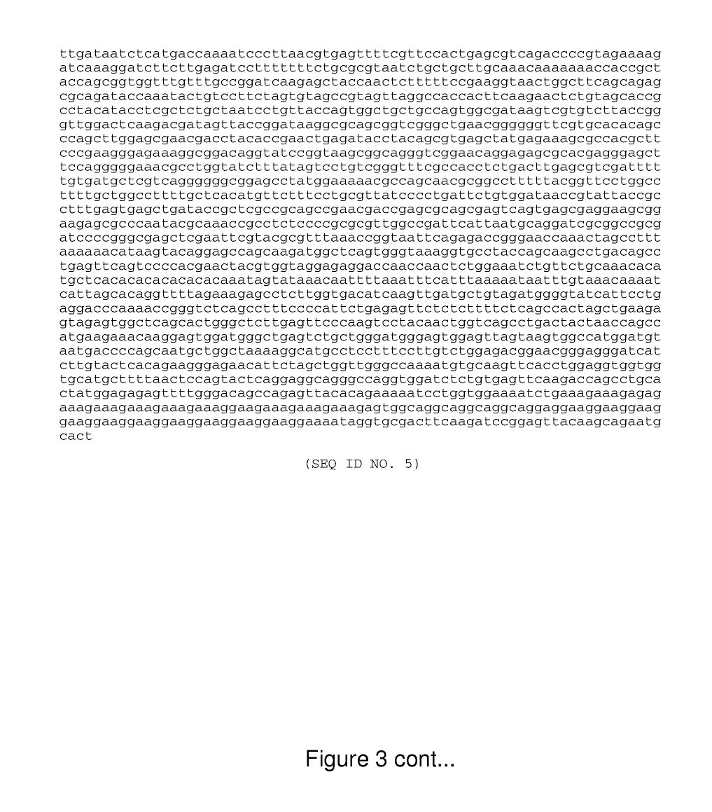

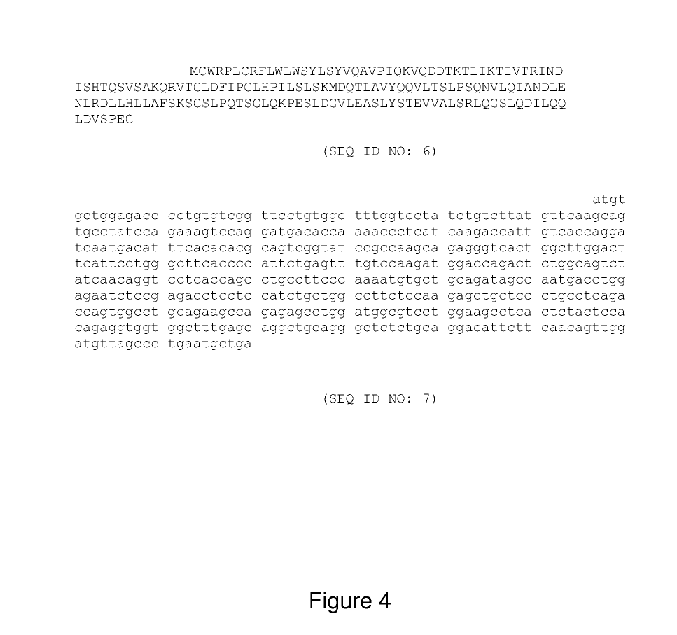

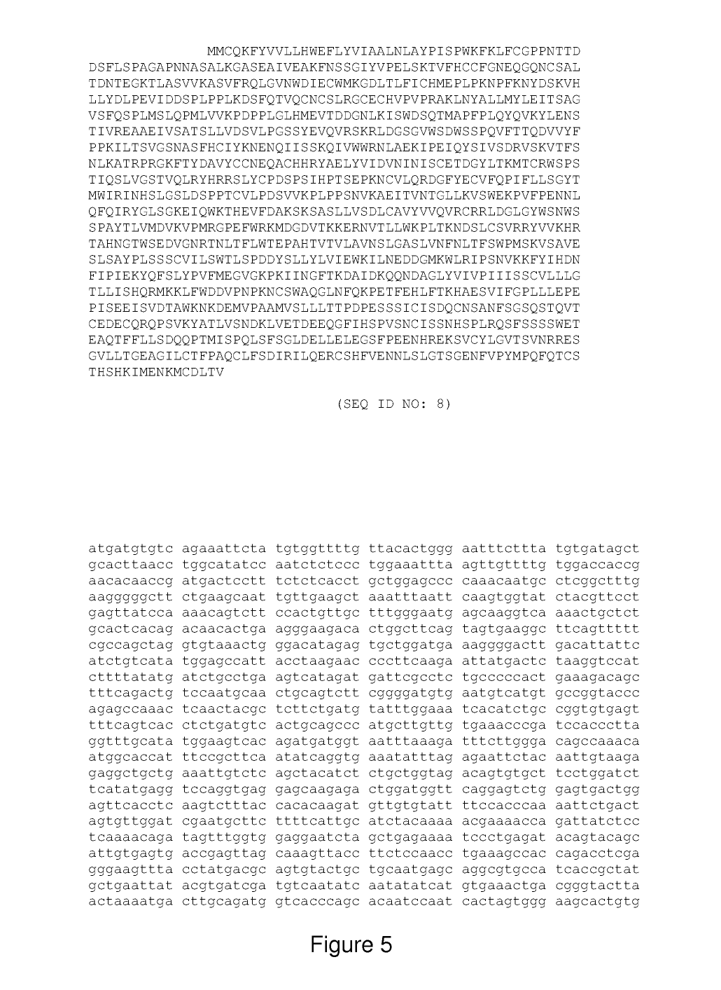

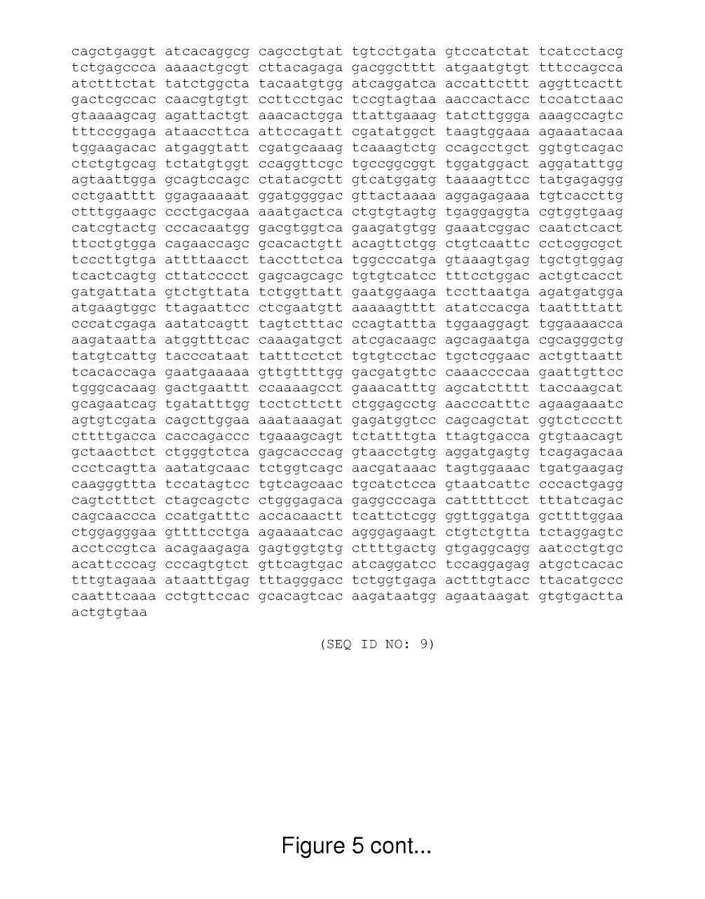

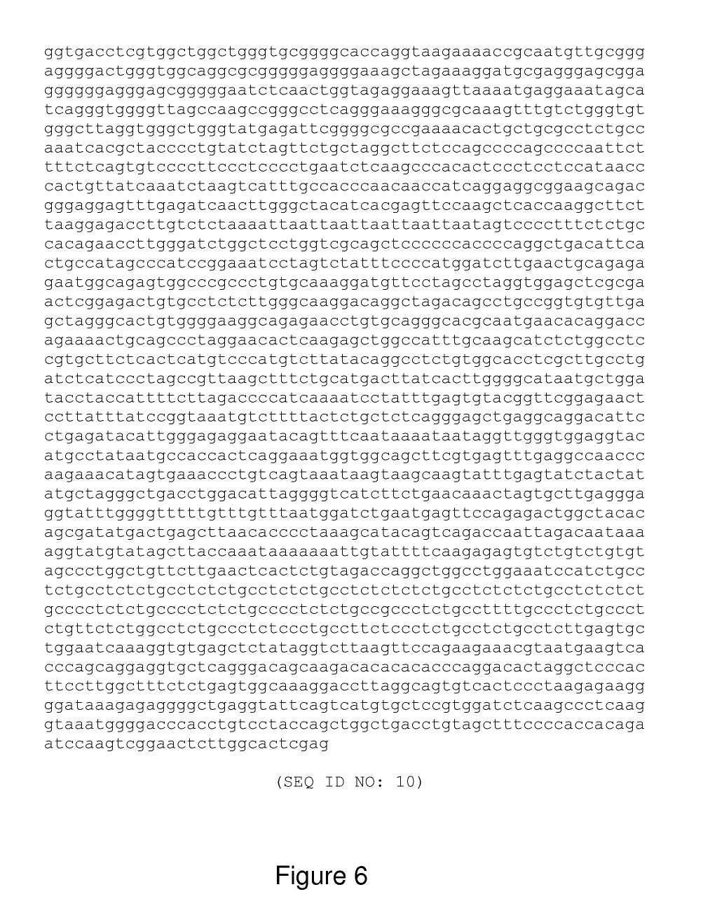

The amplified DNA was cloned into a TA cloning vector pCR2.1, and the resulting plasmid containing the OB-Re cDNA was named pCR2.1-OBRe. The insert was fully sequenced and checked against the published sequence for Ob-Re (accession number U49110). OB-Re cDNA was then transferred into a mammalian expression vector pCMV5-Flag by cloning a 2.4 kb (NotI)-HindIII fragment from pCR2.1-OBRe into pCMV5-Flag's (EcoRI)-HindIII sites. Restriction enzymes inside parenthesis indicate additional treatment that renders the sites blunt. The new plasmid was named pCMV5-Flag-OBRe. The 2.4 kb coding region for Flag-Ob-Re between the restriction sites of (SacI) and (XbaI) was cloned into pThy1-wp1's (XhoI) site (modified from pThy1 mini gene vector, which was a generous gift of Dr. M. Goedert, MRC Laboratory Cambridge UK) to create pThy1-Flag-OBRe. The modified vector pThy1-wp1 (see FIG. 3) contains the Thy1 genes without exon 3 and flanking introns, and additional restriction sites inserted into the original EcoRI site to allow subsequent linearization and release of the transgene. To enhance the expression of the Flag-OBRe in the transgenic vector, a 1 kb HindIII-(StuI) DNA fragment containing hGH poly-Adenylation tail and SV40 enhancer from pCMV5-Flag-OBRe was cloned into pThy1-Flag-OBRe's HindIII-(SalI) sites. This, however, deleted a 3.5 kb HindIII-HindIII fragment in pThy1-Flag-OBRe, which was inserted back to generate the final transgenic construct pThy1-Flag-OBRe-hGH-SV40.

In the transgenic vector, the Flag-OBRe coding sequence is followed by a polyadenylation signal from human growth hormone and the SV40 enhancer to enhance transcription. The addition of the hGH poly-A tail and SV40 enhancer was shown previously to enhance the transgene expression level. The Thy1-gene vector in the final transgenic DNA construct lacks exon 3 and the flanking introns. The remaining promoter specifies the downstream Flag-OBRe coding sequence to express only in neurons.

### Example 2

### Generation of Transgenic Mice

Transgenic mice were generated by injection of gel-purified pThy1-Flag-OBRe-hGH-SV40 (without the vector backbone) into fertilized oocytes using standard procedures.

Collection of Fertilized Embryos from Superovulated Female Mice

Fertilized ova for the microinjection of DNA were obtained from female mice that were mated with proven male breeders. To increase the yield and quality of eggs, female mice were superovulated with gonadotrophin injections. Fertilized embryos were harvested after dissection of the oviduct from newly plugged mice. After injection of the transgene pThy1-Flag-OBRe-Flag-SV40, groups of 20 embryos were re-implanted into the oviduct of pseudopregnant female recipients. These mice would then give birth 19-20 days after implantation. Out of two rounds of injections, some 70 pups were born. Three weeks after birth, mice were weaned, and male and female mice were separated. Transgene integration was assessed by PCR of genomic DNA extracted from mouse tail. The following oligonucleotide pair was used:

| | | |
|---|---|---|
| wh151 | (GACTACAAGGACGATGACGA) | [SEQ ID No. 13] |
| and | | |
| wh152 | (TCCCCTTTCATCCAGCACTC). | [SEQ ID No. 14] |

The expected PCR fragment size from transgenic mice is 460 bases. Out of the 70 pups, we have 8 positive mice as reported by PCR genotyping. The 8 positive founder mice are in breeding to establish individual colonies. After transgenic lines from each founder mouse are established the protein expression levels and stability of transgene transmission are analyzed.

### Example 3

### Use of Transgenic Non-Human Animals to Identify Compounds Useful for the Treatment of Obesity and/or Alzheimer's Disease

Inhibition of the following negative regulators of leptin signaling, PTP1B, SOCS3 and PTEN, may enhance one or more of the leptin signaling pathways.

### PTP1B Inhibitors

PTP1B is a negative regulator of OBRb-Jak2. Inhibition of PTP1B enhances leptin signaling. Some of the compounds, including benzofuran, benzothiophene biphenyl, and vanadate, have been shown to be potent inhibitors of PTP1B.

### SOCS3 Inhibitors

SOCS3 is a negative regulator of STAT3, a key signaling molecule downstream of leptin-leptin receptor activation. Inhibition of SOCS3 increases the level/activity of STAT3, and in turn enhances leptin signaling. SOCS3 activity may be inhibited by specific antibodies raised against it, or by a compound/molecule that prevents its phosphorylation.

PTEN Inhibitors

PTEN promotes PIP3 to PIP2, which indirectly inhibits PI3K and AKt activation, a key leptin signaling pathway. Inhibitors of PTEN therefore increase PIP3, and subsequent PI3K/AKt activity, and thus enhance leptin signaling. Some inhibitors are commercially available, for example, Dipotassium Bisperoxo(5-hydroxypyridine-2-carboxyl)oxovanadate and Potassium Bisperoxo(1,10-phenanthroline)oxovanadate from Calbiochem.

Compounds/molecules described above are tested in two steps: first, their anti-obesity effects are tested, and second, their neuroprotection effects are tested.

For anti-obesity effects, two sets of experiment are performed.

(1) The effects of molecules/compounds on normal wild type animal are tested by administering the molecules/compounds and observing whether the animals that are given the compounds/molecules are more resistant to diet-induced obesity than those that are given control or placebo compounds/molecules. Diet-induced obesity refers to feeding the animals under experiment with high-fat content diet. Animals usually develop obesity after 10-14 weeks of high fat diet feeding.

(2) The effects of molecules/compounds on transgenic animals that over-expresses Flag-Ob-Re in a neuron-specific manner are compared with their control litter mates (the control litter mates are non-transgenic). This involves testing (a) whether transgenic animals that are given the compounds/molecules are more resistant to diet-induced obesity than those transgenic animals that are given control or placebo compounds/molecules; (b) whether transgenic animals that are given the compounds/molecules are similar to their control littermates that are given the same compounds/molecules in resisting diet-induced obesity; and (c) whether transgenic animals that are given the compounds/molecules are more resistant to diet-induced obesity than their control littermates that are given the control/placebo compounds/molecules.

Lead compounds/molecules are identified that have anti-obesity effects in (1), which are then used for step (2) of the testing.

For neuroprotective effects, the compounds/molecules identified with anti-obesity properties are tested for neuroprotective effects in the following experiments:

(1) The effects of molecules/compounds on normal wild type animal are compared. Animals that are given the compounds/molecules are tested to see whether they perform better in behavioral tests, such as learning/memory tests, than those that are given control or placebo compounds/molecules. The behavioral tests may include typical water maze test, Y-maze test or their variants.

(2) The effects of molecules/compounds on transgenic animals that over-express Flag-Ob-Re in a neuron-specific manner are compared with their control litter mates (the control litter mates are non-transgenic). The animal are tested to see (a) whether transgenic animals that are given the compounds/molecules perform better in behavioral tests than those transgenic animals that are given control or placebo compounds/molecules; (b) whether transgenic animals that are given the compounds/molecules are similar to their control littermates that are given the same compounds/molecules in performing behavioral tests; and (c) whether transgenic animals that are given the compounds/molecules perform better in behavioral tests than their control littermates that are given the control/placebo compounds/molecules.

Besides the behavioral tests describe above, more detailed electrophysiological/optical imaging methods may be used to compare synaptic functions between various groups as described above. Furthermore, histological and immunological analysis may be performed to assess the effects of the compounds in preventing the formation of plaque/neurofibrillary tangles in groups receiving compounds/molecules as compared with those groups receiving placebo compounds/molecules.

### REFERENCES

All references in this specification are incorporated by reference.
1. Ahima R S, Flier J S, Annu. Rev. Physiol (2000) 62:413-37
2. Baile C A, Della-Ferra M A, Martin R J, Annu. Rev. Nutr. (2000) 20:105-27
3. Bonini N M, Fortini M E, Annu. Rev. Neurosci. (2003) 26:627-56
4. Fewlass D C, Noboa K, Pi-Sunyer F X, Johntson J M, Yan S D, Tezapsidis N, FASEB J. (2004) 18:1870-78
5. Gao Q, Wolfgang M J, Neschen S, Morino K, Horvath T L, Shulman G I, Fu X-Y, Proc. Natl. Acad. Sci. U.S.A. (2004) 101:4661-4666
6. Heintz N, Zoghbi H Y, Annu. Rev. Physiol. (2000) 62:779-802
7. Price D L, Sisodia S S, Annu. Rev. Neurosci. (1998) 21:479-505
8. Robinson S W, Dinulescu D M, Cone R D (2000) 34:687-745
9. Stokin G B, Goldstein L S B, Annu. Rev. Biochem. (2006) 75:607-27

1. A transgenic non-human animal having a nucleic acid inserted in its genome, wherein the presence of the inserted nucleic acid in the genome of the animal results in expression of an agent, which agent is encoded by a nucleotide sequence in the genome of the animal, and wherein the agent inhibits the ability of a leptin to activate an Ob-Rb receptor.

2. A transgenic non-human animal according to claim 1, wherein the agent inhibits binding of the leptin to the Ob-Rb receptor.

3. A transgenic non-human animal having a nucleic acid inserted in its genome, wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor.

4. A transgenic non-human animal according to claim 3, wherein the agent inhibits the ability of the leptin to activate the Ob-Rb receptor.

5. A transgenic non-human animal according to claim 1 or claim 3, wherein the inserted nucleic acid is present at a position in the genome of the animal, at which position the inserted nucleic acid does not occur naturally.

6. A transgenic non-human animal according to claim 1 or claim 3, wherein the presence of the inserted nucleic acid in the genome of the animal results in over-expression of the agent.

7. A transgenic non-human animal according to claim 1 or claim 3, wherein the inserted nucleic acid comprises the nucleotide sequence encoding the agent.

8. A transgenic non-human animal according to claim 1 or claim 3, wherein the inserted nucleic acid comprises a heterologous construct.

9. A transgenic non-human animal according to claim 8, wherein the heterologous construct comprises a nucleotide sequence encoding the agent and a regulatory element, which regulatory element is operably linked to the nucleotide sequence encoding the agent.

10. A transgenic non-human animal according to claim 1 or claim 3, wherein the agent is specifically expressed in the nervous system.

11. A transgenic non-human animal according to claim 1 or claim 3, wherein the agent specifically binds to the leptin.

12. A transgenic non-human animal according to claim 11, wherein binding of the agent to the leptin prevents binding of the leptin to the Ob-Rb receptor.

13. A transgenic non-human animal according to claim 1 or claim 3, wherein the agent is not membrane-bound.

14. A transgenic non-human animal according to claim 1 or claim 3, wherein the agent comprises the leptin binding domain of a leptin receptor.

15. A transgenic non-human animal according to claim 1 or claim 3, wherein the agent is Ob-Re.

16. A transgenic non-human animal according to claim 1 or claim 3, wherein the animal is a rodent.

17. A transgenic non-human animal according to claim 1 or claim 3, wherein the animal is a mouse.

18. A transgenic non-human animal according to claim 1 or claim 3, wherein the animal is a model for obesity and/or neurodegenerative disease.

19. A transgenic non-human animal according to claim 1 or claim 3, wherein the animal has an increased susceptibility for obesity and/or neurodegenerative disease.

20. A transgenic animal according to claim 18, wherein the neurodegenerative disease is Alzheimer's disease.

21. A method of producing a transgenic non-human animal, comprising the steps of:
(i) inserting a nucleic acid into the genome of a non-human animal germ line cell; and
(ii) generating a non-human animal from said non-human animal germ line cell;
wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor.

22. A method according to claim 21, wherein the agent inhibits the ability of the leptin to activate the Ob-Rb receptor.

23. A method according to claim 21, wherein the nucleic acid is inserted at a position in the genome of the animal, at which position the inserted nucleic acid does not occur naturally.

24. A method according to claim 21, wherein the presence of the inserted nucleic acid in the genome of the animal results in over-expression of the agent.

25. A method according to claim 21, wherein the inserted nucleic acid comprises the nucleotide sequence encoding the agent.

26. A method according to claim 21, wherein the inserted nucleic acid comprises a heterologous construct.

27. A method according to claim 26, wherein the heterologous construct comprises a nucleotide sequence encoding the agent and a regulatory element, which regulatory element is operably linked to the nucleotide sequence encoding the agent.

28. A method according to claim 21, wherein the agent is specifically expressed the nervous system.

29. A method according to claim 21, wherein the agent specifically binds to the leptin.

30. A method according to claim 29, wherein binding of the agent to the leptin prevents binding of the leptin to the Ob-Rb receptor.

31. A method according to claim 21, wherein the agent is not membrane bound.

32. A method according to claim 21, wherein the agent comprises the leptin binding domain of a leptin receptor.

33. A method according to claim 21, wherein the agent is Ob-Re.

34. A method according to claim 21, wherein the germ-line cell is an oocyte, egg, or embryonic stem cell.

35. A method according to claim 21, wherein the animal is a model for obesity and/or neurodegenerative disease.

36. A method according to claim 21, wherein the animal has an increased susceptibility for obesity and/or neurodegenerative disease.

37. A method according to claim 35 or claim 36, wherein the neurodegenerative disease is Alzheimer's disease.

38. A method according to claim 21, wherein the transgenic non-human animal is a rodent.

39. A method according to claim 38, wherein the rodent is a mouse.

40. A method of identifying and/or obtaining a compound useful in the treatment of obesity, comprising the steps of:
(i) administering a test compound to a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor; and
(ii) determining the effect of the test compound on the susceptibility of the animal to gain weight.

41. A method according to claim 40, wherein a reduction in the susceptibility of the transgenic non-human animal to gain weight in the presence of the test compound relative to the absence of the test compound is indicative that the compound is useful in the treatment of obesity.

42. A method of identifying and/or obtaining a compound that represses the development of obesity, comprising the steps of:
(i) providing a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor, and wherein the animal has a capacity to gain weight;
(ii) administering a test compound to the animal, and
(iii) monitoring the weight gain in the animal.

43. A method according to claim 42, wherein a reduction in the rate of weight gain in the non-human animal in the presence of the test compound relative to the absence of the test compound is indicative that the test compound represses the development of obesity.

44. A method according to claim 41 or claim 43, comprising the step of identifying the test compound as useful in the treatment of obesity.

45. A method of identifying and/or obtaining a compound useful in the treatment of neurodegenerative disease, comprising the steps of:
(i) administering a test compound to a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, and wherein the agent inhibits binding of a leptin to an Ob-Rb receptor; and
(ii) determining the effect of the test compound on neurodegeneration.

46. A method according to claim 45, wherein a reduction in the susceptibility of the non-human animal to neurodegeneration in the presence of the test compound relative to the absence of the test compound is indicative that the compound is useful in the treatment of neurodegenerative disease.

47. A method of identifying and/or obtaining a compound that represses the development of neurodegenerative disease, comprising the steps of:
(i) providing a transgenic non-human animal, which animal has a nucleic acid inserted in its genome, wherein the presence of the inserted nucleic acid in the genome of the animal results in expression of an agent, which agent is encoded by a nucleotide sequence in the genome of the animal, wherein the agent inhibits binding of a leptin to an Ob-Rb receptor, and wherein the animal has a capacity for neurodegeneration;
(ii) administering a test compound to the animal, and
(iii) monitoring the development of neurodegeneration in the animal.

48. A method according to claim 47, wherein monitoring the development of neurodegeneration comprises monitoring the accumulation of amyloid plaque formation and/or accumulation of Aβ in the brain of the animal.

49. A method according to claim 47, wherein a reduction in the development of neurodegeneration in the animal in the presence of the test compound relative to the absence of the test compound is indicative that the compound represses the development of neurodegenerative disease.

50. A method according to claim 46 or claim 49, comprising the step of identifying the test compound as useful in the treatment of neurodegenerative disease.

51. A method according to claim 45 or claim 47, wherein the neurodegenerative disease is Alzheimer's disease.

52. A method according to any one of claims 40, 42, 45 or 47, wherein the transgenic non-human animal is a rodent.

53. A method according to claim 52, wherein the transgenic non-human animal is a mouse.

54. Use of a transgenic non-human animal, as defined in claim 1 or claim 3, in testing compounds for their ability to repress the development of obesity and/or neurodegenerative disease.

55. A therapeutic composition effective in the prevention or treatment of obesity and/or neurodegenerative disease comprising a compound, as identified by a method as defined in any one of claims 40, 42, 45 or 47, and a pharmaceutically acceptable diluent, excipient and/or vehicle.

56. A nucleic acid comprising a nucleotide sequence encoding the Thy-1 promoter operably linked to a nucleotide sequence encoding Ob-Re.

57. A transgenic animal according to claim 19, wherein the neurodegenerative disease is Alzheimer's disease.
